(19) United States
(12) Patent Application Publication
(10) Pub. No.: US 2016/0304459 A1
(43) Pub. Date: Oct. 20, 2016

(54) NOVEL COMPOUND, PRODUCTION METHOD THEREFOR, AND APPLICATION THEREFOR

(71) Applicants: TOKYO UNIVERSITY OF PHARMACY & LIFE SCIENCES, Tokyo (JP); KOKUSAN CHEMICAL CO., LTD., Tokyo (JP)

(72) Inventors: Yoshio HAYASHI, Kanagawa (JP); Akihiro KAJIYAMA, Aichi (JP); Akihiro TAGUCHI, Tokyo (JP); Kentarou FUKUMOTO, Tokyo (JP)

(73) Assignees: TOKYO UNIVERSITY OF PHARMACY & LIFE SCIENCES, Tokyo (JP); KOKUSAN CHEMICAL CO., LTD., Tokyo (JP)

(21) Appl. No.: 15/026,061

(22) PCT Filed: Oct. 2, 2014
(86) PCT No.: PCT/JP2014/076397

§ 371 Date: Jun. 17, 2016

(30) Foreign Application Priority Data
Oct. 4, 2013 (JP) 2013-209166

(51) Int. Cl. C07D 213/82; C08G 65/337; C07K 7/08; C07K 1/06; C07D 207/16; C07K 7/16; C07K 7/06; C07K 1/04

(52) U.S. Cl. CPC C07D 213/82; C07K 7/06; C08G 65/337; C07K 1/042; C07K 1/067; C07D 207/16; C07K 7/16; C07K 7/08

(57) ABSTRACT

[Problems] To provide a novel peptide synthesis technique that is completely different than heretofore, and to provide a novel compound that enables the synthesis/creation of a novel artificial functional protein and the synthesis/creation of a novel functional peptide, as well as a method for producing the same. [Solution] A compound represented by formula (I) or a salt thereof.

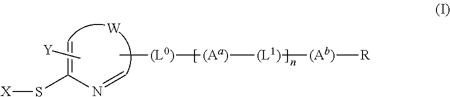

### TECHNICAL FIELD

The present invention relates to a novel compound, a method for producing the same, and a use thereof. More specifically, the present invention provides a compound having a sulfenylpyridine structure supported on a polymeric support, a method for producing this compound, a novel peptide synthesis technique using this compound, and the like.

### BACKGROUND ART

Creating a labeled form using a labeling substance to bond to a biomolecule is often used in fields such as biology, molecular biology, and the like in the analysis, detection, and the like of biomolecules such as proteins, peptides, nucleic acids, and the like. Biotin labeling is a concrete example of molecular labeling. Systems that take advantage of the strong affinity of avidin and biotin are being developed to raise the sensitivity in various assay systems and in the purification of physiologically active substances. A biotin labeling substance is thus essential in this system (Non-patent Reference 1).

A labeled molecule containing a biotin label is generally introduced using a labeling reagent having reactivity to the several functional groups in a physiologically active substance. Examples of these functional groups include amino groups, hydroxyl groups, imidazolyl groups, thiol groups, and the like. Alternatively, there are also reagents capable of selectively labeling any one functional group. An example is a reagent that selectively labels SH groups. Selectively labeling SH groups means selectively labeling cysteine residues in the case of peptides and proteins. The SH group of cysteine and disulfide formed by cysteines are known to greatly affect the steric structure of proteins and enzyme activity of proteins, and identifying the position thereof is important when analyzing the structure or activity of proteins. In fact, a method is known of labeling the SH groups in a protein using a fluorescent substance that bonds to SH groups and identifying their locations.

Labeling is not limited to the application to cysteine and derivatives thereof; a method is known of measuring the activity of cyclin-dependent kinase (referred to hereinafter as CDK) as an example of the labeling of SH groups (Patent Reference 1). In this measurement method, SH groups are first introduced into a substrate of CDK using CDK and adenosine 5′-O-(3-thiotriphosphate). Next, the SH groups introduced into the substrate are labeled by a labeling substance that bonds selectively to SH groups. Measurement of the CDK activity is completed by measuring the labeled substrate.

The following properties are required for these reagents to be used to selectively label SH groups. First, it must label only SH groups and not affect other functional groups. Second, it must be possible to isolate and purify the labeled molecule alone, by a simple procedure from labeling to purification. The accuracy of site identification and activity measurement, which are the original purposes, is harmed when a reagent not only labels SH groups but also acts on other functional groups or structures. A number of reagents characterized by the selective labeling of SH groups have been reported recently (Patent References 2 and 3).

However, labeling procedures by these reagents often use excessive labeling reagent, and unreacted reagent or degradation products of the reagent often remain. This usually necessitates a procedure to remove the excess labeling reagent and byproducts after the labeling reaction.

When the labeling target is a polymer such as a protein or antibody, gel filtration utilizing the difference in molecular weight or (centrifugal) ultrafiltration by a membrane filter can be utilized to remove the excess labeling reagent introduced. When labeling low-molecular compounds, however, these techniques cannot be applied, and more complex purification by chromatography or the like is required to remove the residues of reagent having a similar molecular weight.

A simpler labeling technique is therefore desired in screening that requires labeling of low-molecular organic compounds.

Thus, there have been various problems with conventional reagents used to selectively label SH groups.

In view of the above problems of the prior art, the inventors supported a 2-disulfanylpyridine skeleton which selectively forms asymmetrical disulfide bonds with free SH groups on a solid support and created a compound that makes it possible to introduce a labeling substance efficiently using a combination of solid-phase chemistry techniques with conventional molecular labeling techniques (Patent Reference 4, Non-patent Reference 2). They discovered that using this compound as an SH selective labeling reagent to label compounds having SH groups by a labeling substance makes it possible to establish a technique for labeling low-molecular compounds without going through complicated purification measures.

### PRIOR ART REFERENCES

### Patent References

Patent Reference 1: Japanese Patent Application Publication No. 2002-335997
Patent Reference 2: Japanese Patent Application Publication No. 2004-530885
Patent Reference 3: Japanese Patent Application Publication No. 2010-51289
Patent Reference 4: Japanese Patent Application Publication No. 2012-117981

### Non-Patent References

Non-Patent Reference 1: Chemical Encyclopedia 1, Kyoritsu (Sep. 10, 1967).
Non-Patent Reference 2: Development of a solid-supported biotinylation reagent for efficient biotin labeling of SH groups on small molecules. K. Fukumoto, A. Kajiyama, Y. Hayashi, et al., Tetrahedron Lett., 53(5), 535-538 (2012).

### SUMMARY OF THE INVENTION

### Problems to be Solved by the Invention

It is an object of the present invention to provide a novel peptide synthesis technique completely different from the past having the advantage of not requiring purification and to provide a novel compound that enables the synthesis/creation of a novel artificial protein and the synthesis/creation of a novel functional peptide, or an organic compound not restricted to peptides, and a method for producing the same.

### Means Used to Solve the Above-Mentioned Problems

The compound disclosed in Patent Reference 4 is useful as an SH selective labeling reagent, but is itself not intended to be used as a novel peptide synthesis technique.

Through pursuing additional research, the present inventors focused on 3-nitro-2-chlorosulfenylpyridine having the ability to form S—S bonds, created a compound having a chlorosulfenylpyridine structure supported on a resin, and discovered that, unexpectedly, the use of this compound makes it possible to successively connect many different peptide fragments by a very simple method without going through purification processes, and thereby achieved the present invention.

Specifically, the present invention provides:

[1] A compound represented by formula (I), or a salt thereof.

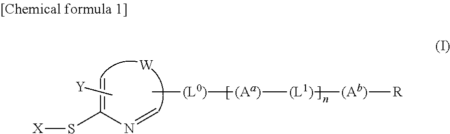

(In the formula,
W, together with other ring member atoms, forms a nitrogen-containing heterocycle selected from pyridine, pyrazine, imidazole, oxazole, thiazole, quinoline, isoquinoline, quinoxaline, phenanthroline, pteridine, or azocine,
X represents a halogen atom selected from fluorine, chlorine, bromine, or iodine,
Y represents a hydrogen atom or electron-withdrawing substituent present on the nitrogen-containing heterocycle,
R represents a polymeric support,
L⁰, L¹ may each be present independently and, when present, represent linkers having a chemically stable structure,
A^{a}, A^{b} may each be present independently and, when present, represent functional groups connecting L⁰-L¹, L¹-R, respectively, and
n represents an integer of 0-10.)

[2] The compound according to [1], or a salt thereof, wherein A^{a}, A^{b}, when present, are each independently selected from the group consisting of alkenes, alkynes, carbonyls, esters, ethers, oxyalkylenes, amides, ureas, hydrazines, triazoles, sulfones, sulfoxides, sulfonic acid esters, sulfonamides, sulfinic acid esters, sulfinamides, piperidines, and dioxanes.

[3] The compound according to [1] represented by formula (II), or a salt thereof, wherein the nitrogen-containing heterocycle is a pyridine ring, L¹ is not present, A^{a} is an amide group, A^{b} is not present, and n is 1.

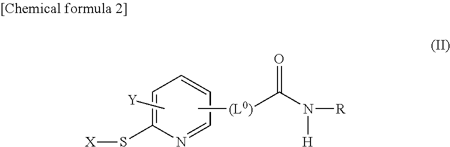

(In the formula, X, Y, R, and L⁰ are as defined in formula (I).)

[4] The compound according to [1] represented by formula (II-a), or a salt thereof, wherein the nitrogen-containing heterocycle is a pyridine ring, A^{a} is an amide group, A^{b} is an amide group, and n is 1-5.

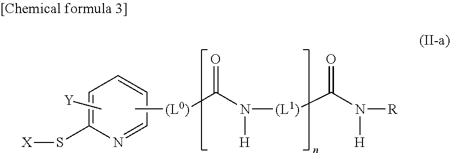

(In the formula, X, Y, R, L⁰, and L¹ are as defined in formula (I).)

[5] The compound according to any one of [1]-[4], or a salt thereof, wherein the electron-withdrawing substituent is a nitro group, trifluoromethyl group, or halogen.

[6] The compound according to any one of [1]-[5], or a salt thereof, wherein L⁰ and L¹, when present, each independently are selected from the group consisting of straight or branched C1-C20 alkylenes, C2-C20 alkenylenes, C2-C20 alkynylenes, cycloalkylenes having 3-20 carbon atoms, cycloalkenylenes having 3-20 carbon atoms, arylenes, monocyclic heteroarylenes, heterocycles, amines, amides, ethers, esters, sulfides, ketones, polyethylene glycol chains, and groups represented by formula (a)

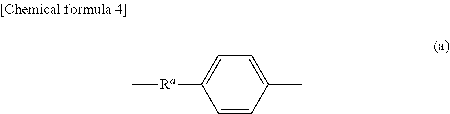

(in the formula, R^{a} represents an optionally substituted C1-C15 alkylene), and these alkylenes, alkenylenes, alkynylenes, cycloalkylenes, cycloalkenylenes, arylenes, and monocyclic heteroarylenes are optionally substituted.

[7] The compound according to any one of [1]-[6], or a salt thereof, wherein R is a polymeric support used in a solid-phase synthesis method.

[8] The compound according to [7], or a salt thereof, wherein R is selected from the group consisting of polystyrene, polypropylene, polyethylene, polyether, polyvinyl chloride, dextran, acrylamide, polyethylene glycol, copolymers and crosslinked forms thereof, magnetic beads, and combinations thereof.

[9] An SH group selective reactive solid-phase supported reagent containing the compound according to any one of [1]-[8], or a salt thereof.

[10] A method for producing a compound represented by formula (IV), wherein the method comprises reacting a compound represented by formula (I)

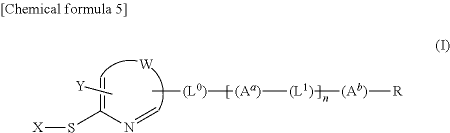

(in the formula, W, together with other ring member atoms, forms a nitrogen-containing heterocycle selected from pyridine, pyrazine, imidazole, oxazole, thiazole, quinoline, isoquinoline, quinoxaline, phenanthroline, pteridine, or azocine,
X represents a halogen atom selected from fluorine, chloride, bromine, or iodine,
Y represents a hydrogen atom or electron-withdrawing substituent,
R represents a polymeric support,
L⁰, L¹, when present, represent linkers having a chemically stable structure,
A^{a}, A^{b}, when present, represent functional groups connecting L⁰-L¹, L¹-R, respectively, and
n represents an integer of 0-10)
with a compound represented by formula (III)

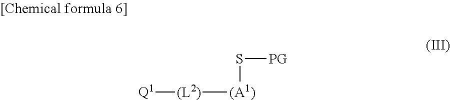

(in the formula,
Q¹ represents an organic compound,
L², when present, represents a linker having a chemically stable structure,
A¹, when present, represents a functional group having S-PG, PG represents an SH group protecting group or hydrogen atom) to produce a compound represented by formula (IV)

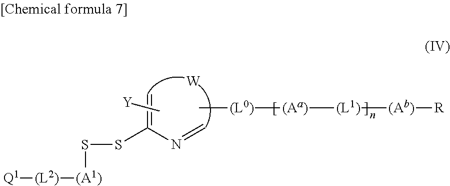

(in the formula, W, Y, R, L⁰, L¹, A^{a}, A^{b}, and n are as defined in formula (I), and Q¹, L², and A¹ are as defined in formula (III)).

[11] The method according to [10] wherein L² is selected from the group consisting of straight or branched C1-C10 alkylenes, C2-C10 alkenylenes, C2-C10 alkynylenes, cycloalkylenes having 3-10 carbon atoms, cycloalkenylenes having 3-10 carbon atoms, arylenes, monocyclic heteroarylenes, heterocycles, amines, amides, ethers, esters, sulfides, ketones, polyethylene glycol chains, polyamides, and groups represented by formula (a)

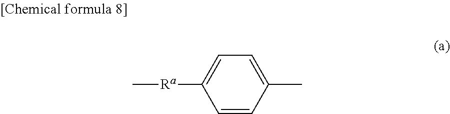

(in the formula, R^{a} represents an optionally substituted C1-C15 alkylene), and these alkylenes, alkenylenes, alkynylenes, cycloalkylenes, cycloalkenylenes, arylenes, and monocyclic heteroarylenes are optionally substituted.

[12] The method according to [10] or [11] wherein Q¹ is selected from the group consisting of biological organic compounds, selected from amino acids, peptides, proteins, antibodies, nucleic acid bases, nucleotides or nucleosides, polymer compounds, low-molecular compounds, fluorescent labeling substances, enzyme labeling substances, biotin, chelating agents, and derivatives thereof including isotopes.

[13] The method according to any one of [10]-[12] wherein the SH group protecting group is selected from t-butyl, trityl, benzhydryl, benzyl, methylbenzyl, dimethylbenzyl, trimethylbenzyl, methoxybenzyl, dimethoxybenzyl, trimethoxybenzyl, nitrobenzyl, acetamidomethyl, 9-fluorenylmethyl, carbonylbenzyloxy, diphenylbenzyl, ethylcarbamoyl, picolyl, sulfonyl, or salts thereof.

[14] A method for producing a compound represented by formula (VI), wherein the method comprises reacting a compound represented by formula (IV)

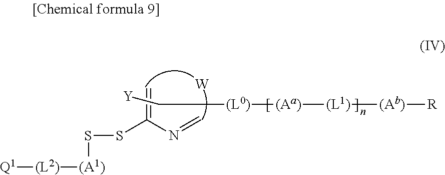

(in the formula,
W, together with other ring member atoms, forms a nitrogen-containing heterocycle selected from pyridine, pyrazine, imidazole, oxazole, thiazole, quinoline, isoquinoline, quinoxaline, phenanthroline, pteridine, or azocine,
Y represents a hydrogen atom or electron-withdrawing substituent,
R represents a polymeric support,
L⁰, L¹, L², when present, represent linkers having a chemically stable structure,
A^{a}, A^{b}, when present, represent functional groups connecting L⁰-L¹, L¹-R, respectively,
A¹, when present, represents a functional group having S-PG,
Q¹ represents an organic compound, and
n represents an integer of 0-10)
with a compound represented by formula (V)

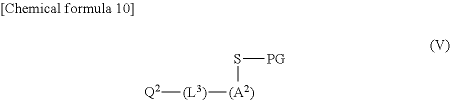

(in the formula,
Q² represents an organic compound,
L³, when present, represents a linker having a chemically stable structure,
A², when present, represents a functional group having S-PG, and
PG represents an SH group protecting group or hydrogen atom) to produce a compound represented by formula (VI)

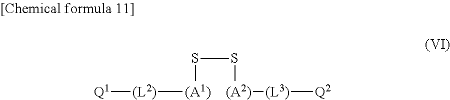

(in the formula, Q¹, Q², L², L³, A¹, and A² are as defined above).

[15] The method according to [14] wherein the electron-withdrawing substituent is a nitro group, trifluoromethyl group, or halogen.

[16] The method according to [14] or [15] wherein L² and L³ are each independently selected from the group consisting of straight or branched C1-C10 alkylenes, C2-C10 alkenylenes, C2-C10 alkynylenes, cycloalkylenes having 3-10 carbon atoms, cycloalkenylenes having 3-10 carbon atoms, arylenes, monocyclic heteroarylenes, heterocycles, amines, amides, ethers, esters, sulfides, ketones, polyethylene glycol chains, polyamides, and groups represented by formula (a)

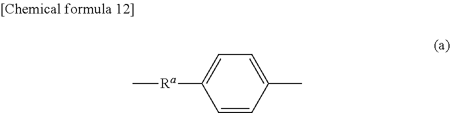

(in the formula, R^{a} represents an optionally substituted C1-C15 alkylene), and these alkylenes, alkenylenes, alkynylenes, cycloalkylenes, cycloalkenylenes, arylenes, and monocyclic heteroarylenes are optionally substituted.

[17] The method according to any one of [14]-[16] wherein Q¹ and Q² are each independently selected from the group consisting of biological organic compounds, selected from amino acids, peptides, proteins, antibodies, nucleic acid bases, nucleotides or nucleosides, polymer compounds, low-molecular compounds, fluorescent labeling substances, enzyme labeling substances, chelating agents, biotin, and derivatives thereof including stable isotopes.

[18] A method for producing a compound represented by formula (II), wherein the method comprises the following steps of:

(a) preparing a compound represented by formula (2) by reacting a compound represented by formula (1) with thionyl chloride, oxalyl chloride, dichloroalkylhydantoin, phosphorus oxychloride, or phosphorus pentachloride,

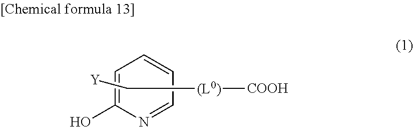

(wherein Y represents a hydrogen atom or electron-withdrawing substituent, and L⁰, when present, represents a chemically stable linker)

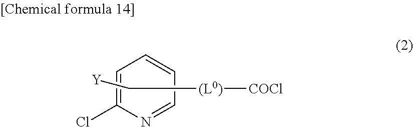

(b) preparing a compound represented by formula (3) by reacting a compound represented by formula (2) with R′OH (wherein R′ represents a C1-C10 alkyl group),

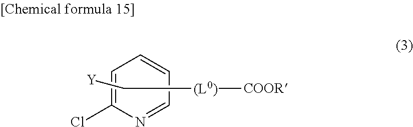

(c) preparing a compound represented by formula (4) by reacting a compound represented by formula (3) with a primary to tertiary alkylthiol under basic conditions,

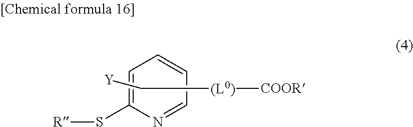

(wherein R″ represents a primary to tertiary carbon serving as a leaving group)

(d) preparing a compound represented by formula (5) by hydrolyzing a compound represented by formula (4) under basic conditions

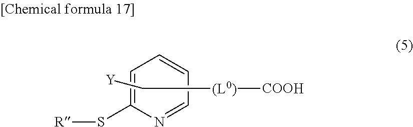

(e) preparing a compound represented by formula (6) by reacting a compound represented by formula (5) with NH₂—R (wherein R represents a polymeric support) in the presence of a base, and

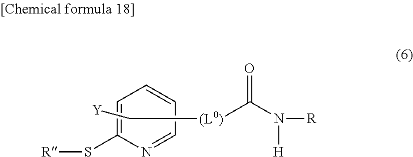

(f) preparing a compound represented by formula (II) by reacting a compound represented by formula (6) with sulfuryl chloride, chlorine gas, phosphorus oxychloride, phosphorus pentachloride, bromine, fluorinated alkyl pyridine, fluorinated quinuclidine, or iodine

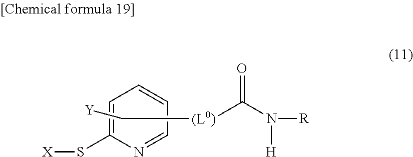

(wherein X represents a halogen atom selected from fluorine, chloride, bromine, or iodine, Y represents a hydrogen atom or electron-withdrawing substituent, R represents a polymeric support, and L⁰, when present, represents a linker having a chemically stable structure).

[19] A method for producing a compound represented by formula (II-a′), wherein the method comprises the following steps of:

(g) preparing a compound represented by formula (8) by reacting a compound represented by formula (7) with NH₂—R (wherein R represents a polymeric support) in the presence of a dehydrocondensing agent,

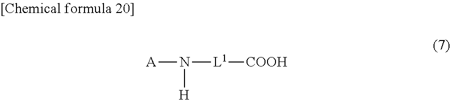

(wherein A represents an amino group protecting group having a urethane structure, and L¹ represents a linker having a chemically stable structure)

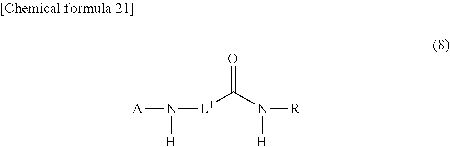

(h) preparing a compound represented by formula (9) by reacting a compound represented by formula (8) with piperidine, diethylamine, dialkylamine, trifluoroacetic acid, hydrochloric acid, or hydrogen chloride or through catalytic hydrogen reduction,

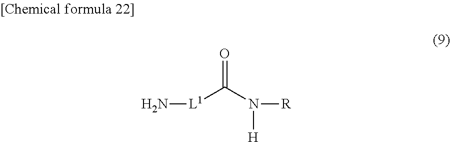

(i) preparing a compound represented by formula (10) by reacting a compound of formula (9) with a compound of formula (7) in the presence of a dehydrocondensing agent,

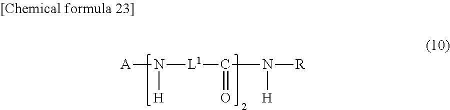

(j) preparing a compound represented by formula (11) by alternately subjecting a compound represented by formula (10) repeatedly to the procedures of steps (h) and (i) n-2 times,

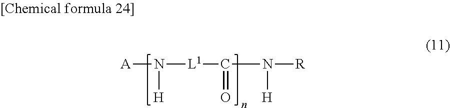

(k) preparing a compound represented by formula (12) by reacting a compound represented by formula (11) with piperidine, diethylamine, dialkylamine, trifluoroacetic acid, hydrochloric acid, or hydrogen chloride or through catalytic hydrogen reduction,

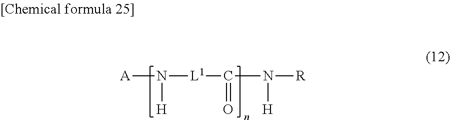

(1) preparing a compound represented by formula (13) by reacting a compound represented by formula (12) with a compound represented by formula (5) in the presence of a dehydrocondensing agent, and

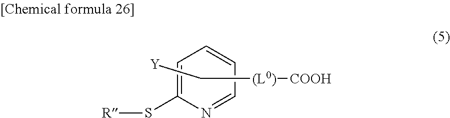

(wherein Y represents a hydrogen atom or electron-withdrawing substituent, L⁰, when present, represents a chemically stable linker, and R″ represents a primary to tertiary carbon serving as a leaving group)

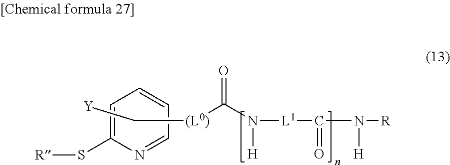

(m) preparing a compound represented by formula (II-a′) by reacting a compound represented by formula (13) with sulfuryl chloride or chlorine gas

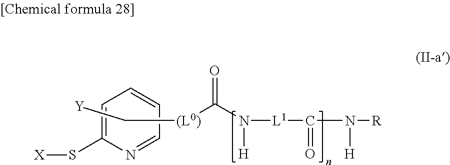

(in the formula, X represents a halogen atom selected from fluorine, chlorine, bromine, or iodine, Y represents a hydrogen atom or electron-withdrawing substituent, R represents a polymeric support, L⁰, when present, represents a chemically stable linker, L¹ represents a linker having a chemically stable structure, and n represents an integer of 1-10).

### Advantages of the Invention

The use of a compound of the present invention in peptide synthesis makes it possible to connect many different peptide fragments by a simple sequential method. Since functional groups of a sulfenylpyridine structure are immobilized on a resin in the compound of the present invention, a high-purity condensed peptide can be obtained from the filtrate merely by filtration without any special purification in each step of disulfide coupling with another peptide. In addition, since the reactivity of the active disulfide formed on the resin is extremely high and selective, there is no need to protect the side chain functional groups of the peptide chains by protecting groups, making it possible to theoretically connect unprotected peptide fragments any number of times and to obtain a so-called train peptide.

The compound of the present invention also makes it possible to provide a novel synthesis technique different from conventional physiologically active peptide synthesis. Specifically, S—S bonds are finally formed after all of the peptide bonds have been connected in conventional peptide synthesis. However, since peptide fragments are connected by S—S bonds from the start when using the compound of the present invention, meaning that specific peptide bonds are formed by intramolecular reaction following disulfide ligation, this makes it possible to provide a new peptide synthesis technique.

Thus, the compound of the present invention makes it possible to provide a novel synthesis technique for a completely new compound that can be called a “train peptide” or “natural peptide.” Moreover, proteins, specifically, ds-proteins (disulfide proteins) and ultimately giant “artificial enzymes” can also be synthesized by connecting secondary structural domains of a protein as small fragment peptides by S—S bonds.

Therefore, the present invention makes it possible to provide a useful technique that makes it possible to create novel molecules in the pharmaceutical and chemical industries.

### BRIEF DESCRIPTION OF THE DRAWINGS

FIG. 1 Synthesis scheme of a train peptide using a compound of the present invention

FIG. 2 Reverse-phase HPLC chart of 90% formic acid aqueous solution of peptide H-Asn-Cys(tBu)-Pro-Leu-Gly-NH₂ in Example 6. The peak at 13.36 min corresponds to the peptide.

FIG. 3 Reverse-phase HPLC chart of reaction solution two hours after the start of the reaction in the synthesis of compound Z¹ in Example 6. This confirmed the disappearance of the peak at 13.36 min.

FIG. 4 Reverse-phase HPLC chart of 50% N,N-dimethylformamide aqueous solution of peptide Fmoc-Cys-Tyr-Ile-Gln-OH in Example 6. The peak at 17.86 min corresponds to peptide Fmoc-Cys-Tyr-Ile-Gln-OH.

FIG. 5 Reverse-phase HPLC chart of reaction solution 30 minutes after the start of the reaction in the synthesis of compound Z² in Example 6. This confirmed the disappearance of the peak corresponding to peptide Fmoc-Cys-Tyr-Ile-Gln-OH at 17.86 min and the appearance of a new peak at 11.86 min.

FIG. 6 Reverse-phase HPLC chart of reaction solution after overnight reaction in the synthesis of compound Z³ in Example 6. The peak at 11.86 min corresponding to compound Z² was not observed, and the appearance of a new peak at 15.99 min was confirmed.

FIG. 7 Reverse-phase HPLC chart of reaction solution after overnight reaction in the synthesis of oxytocin (compound Z) in Example 6. The peak at 15.99 min corresponding to compound Z³ was not observed, and the appearance of a new peak at 12.40 min was confirmed. Furthermore, the peaks detected at 8.82 min and 16.78 min correspond to byproducts derived from reagent degradation products.

FIG. 8 Reverse-phase HPLC chart after 30 minutes of reaction in the synthesis of train peptide compound V¹ in Example 7. The peak at 17.04 min corresponds to compound V¹. The peak seen at 2-3 min corresponds to sodium ascorbate.

### MODE FOR CARRYING OUT THE INVENTION

One embodiment of the present invention is a compound represented by formula (I), or a salt thereof.

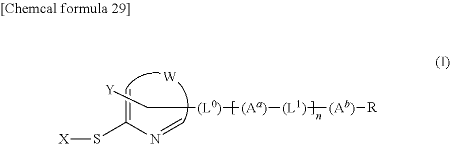

In formula (I), W, together with other ring member atoms, forms a nitrogen-containing heterocycle selected from pyridine, pyrazine, imidazole, oxazole, thiazole, quinoline, isoquinoline, quinoxaline, phenanthroline, pteridine, or azocine, and preferably is pyridine.

In formula (I), X represents a halogen atom selected from fluorine, chlorine, bromine, or iodine, and is preferably chlorine or bromine.

In formula (I), Y represents a hydrogen atom or electron-withdrawing substituent. Preferred as electron-withdrawing substituents are a nitro group, trifluoromethyl group, or halogen (for example, chlorine); more preferred is a nitro group.

In formula (I), L⁰ bonds chemically with the nitrogen-containing heterocycle W and represents a linker having a stable structure. Linkers represented as L⁰ are selected from the group consisting of straight or branched C1-C10 alkylenes, C2-C10 alkenylenes, C2-C10 alkynylenes, cycloalkylenes having 3-10 carbon atoms, cycloalkenylenes having 3-10 carbon atoms, arylenes, monocyclic heteroarylenes, heterocycles, amines, amides, ethers, esters, sulfides, ketones, polyethylene glycol chains, and groups represented by formula (a)

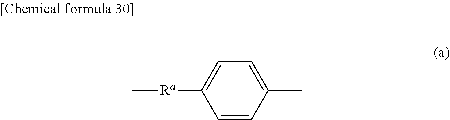

(in the formula, R^{a} represents an optionally substituted C1-C15 alkylene. Here, arbitrary substituents can be selected as substituents; examples include alkyl groups, optionally substituted (for example, alkyl groups, alkoxy groups, halogens, and the like) aryl groups, alkoxy groups, and the like.)

C2-C6 alkylenes and polyethylene glycol chains having a molecular weight of 100-1000 are preferred as L⁰, or L⁰ itself may not be present. When L⁰ is not present, the nitrogen-containing heterocycle W takes on a structure bonded directly with A^{a}.

The above alkylenes, alkenylenes, alkynylenes, cycloalkylenes, cycloalkenylenes, arylenes, and monocyclic heteroarylenes are optionally substituted, and arbitrary substituents can be selected as substituents.

In formula (I), L¹ represents a linker having a chemically stable structure. Linkers represented as L¹ can be selected from the group consisting of straight or branched C1-C10 alkylenes, C2-C10 alkenylenes, C2-C10 alkynylenes, cycloalkylenes having 3-10 carbon atoms, cycloalkenylenes having 3-10 carbon atoms, arylenes, monocyclic heteroarylenes, heterocycles, amines, amides, ethers, esters, sulfides, ketones, polyethylene glycol chains, and groups represented by formula (a)

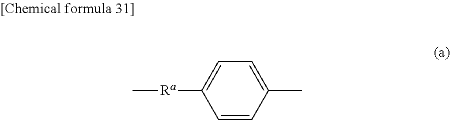

(in the formula, R^{a} represents an optionally substituted C1-C15 alkylene. Here, arbitrary substituents can be selected as substituents; examples include alkyl groups, optionally substituted (for example, alkyl groups, alkoxy groups, and the like) aryl groups, alkoxy groups, and the like.)

L¹ is preferably a C1-C6 alkylene, polyethylene glycol chain having a molecular weight of 100-1000, or group represented by formula (a).

The above alkylenes, alkenylenes, alkynylenes, cycloalkylenes, cycloalkenylenes, arylenes, and monocyclic heteroarylenes are optionally substituted. Examples of substituents include substituted or unsubstituted alkyl groups, substituted or unsubstituted aryl groups, halogens, nitriles; carboxylic acids, sulfonic acids, sulfinic acids, and salts of these. Here, examples of possible substituents of alkyl groups and aryl groups include alkyl groups, aryl groups; carboxylic acids, sulfonic acids, sulfinic acids, and salts of these; amino groups, hydroxyl groups, guanidino groups, alkoxy groups, monocyclic heteroaryls, carbamoyl groups, thiol groups, thioether groups, sulfoxides, sulfones, and the like.

In formula (I), A^{a}, when present, represents a functional group connecting “L⁰-L¹.” Here, when linker L⁰ is not present, A^{a} represents a functional group chemically bonded with the nitrogen-containing heterocycle W. Functional groups represented as A^{a} are selected from the group consisting of alkenes, alkynes, carbonyls, esters, ethers, oxyalkylenes, amides, ureas, hydrazines, triazoles, sulfones, sulfoxides, sulfonic acid esters, sulfonamides, sulfinic acid esters, sulfinamides, piperidines, and dioxanes. Carbonyls, esters, amides, ethers, and oxyalkylenes are preferred as L⁰.

In formula (I), A^{b}, when present, represents a functional group connecting “L¹-R.” Here, when linker L¹ is not present, A^{b} represents a functional group chemically bonded with R. Functional groups represented as A^{b} are selected from the group consisting of alkenes, alkynes, carbonyls, esters, ethers, oxyalkylenes, amides, ureas, hydrazines, triazoles, sulfones, sulfoxides, sulfonic acid esters, sulfonamides, sulfinic acid esters, sulfinamides, piperidines, and dioxanes. Carbonyls, esters, amides, ethers, and oxyalkylenes are preferred as A^{b}.

In formula (I), n represents an integer of 0-10, preferably an integer of 0-5.

In formula (I), R represents a polymeric support, typically a polymeric support used in solid-phase synthesis. Such polymeric supports are selected from the group consisting of polystyrene, polypropylene, polyethylene, polyether, polyvinyl chloride, dextran, acrylamide, polyethylene glycol, copolymers and crosslinked forms of these, magnetic beads, and combinations of these, and polystyrene, polyethylene glycol, and crosslinked forms of polyethylene glycol are more preferred. These polymeric supports may bond to substituents of A^{b} through alkyl groups such as methyl groups and the like.

The form of the resin is more preferably spherical. The preferred average particle size of the resin is 100-400 mesh.

One embodiment of a compound of the present invention is a compound represented by formula (I-a) wherein the nitrogen-containing heterocycle W in formula (I) is a pyridine ring.

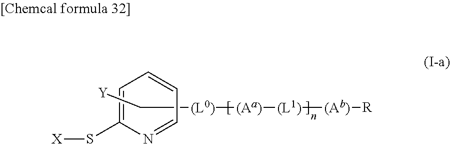

In formula (I-a), X, Y, R, L⁰, L¹, A^{a}, A^{b}, and n are as defined in formula (I).

One embodiment of the compound of the present invention is a compound represented by formula (II) wherein the nitrogen-containing heterocycle W in formula (I) is a pyridine ring, L¹ is not present, A^{a} is an amide, A^{b} is not present, and n is 1.

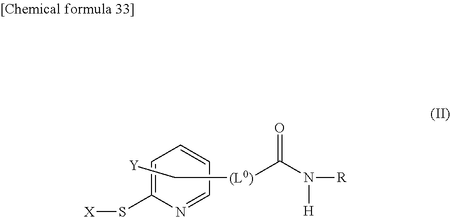

In formula (II), X, Y, R, and L⁰ are as defined in formula (I).

Another embodiment of the compound of the present invention is a compound represented by formula (II-a) wherein the nitrogen-containing heterocycle W is a pyridine ring, A^{a} is an amide, A^{b} is an amide, and n is 1-5.

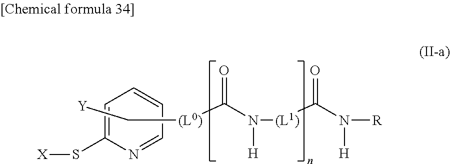

(In the formula, X, Y, R, L⁰, L¹, and n are as defined in formula (I).)

Concrete examples of compounds of formulas (I), (II), and (II-a) of the present invention include, but are not limited to, the following compounds.

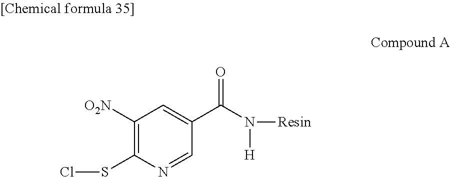

Resin: Crosslinked Polyethylene Glycol (Methyl ChemMatrix® Resin)

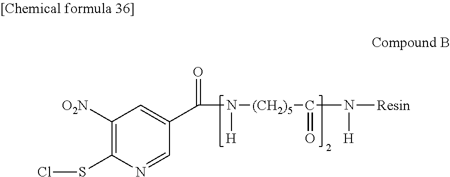

Resin: Crosslinked Polyethylene Glycol (Methyl ChemMatrix® Resin)

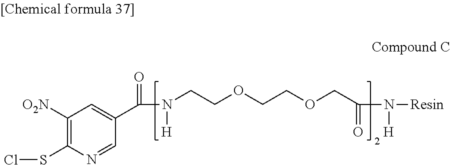

Resin: Crosslinked Polyethylene Glycol (Methyl ChemMatrix® Resin)

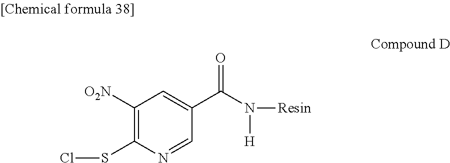

Resin: Polystyrene Resin

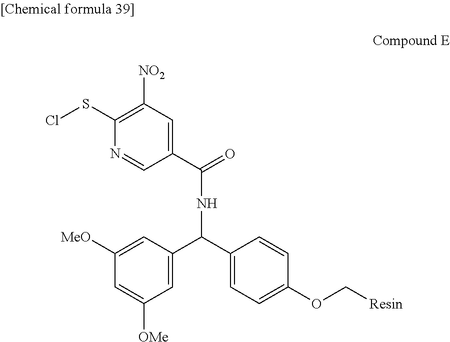

Resin: Polystyrene Resin

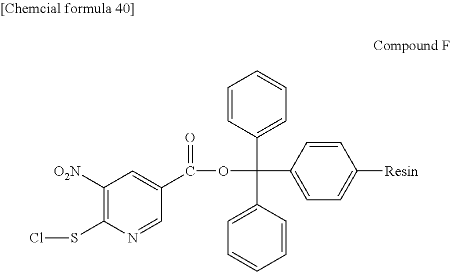

Resin: Polystyrene Resin

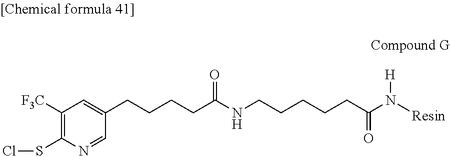

Resin: Polystyrene Resin

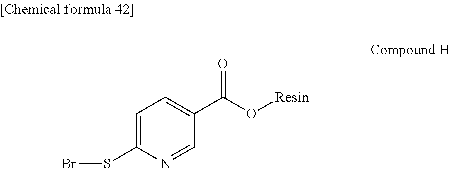

Resin: Polystyrene Resin

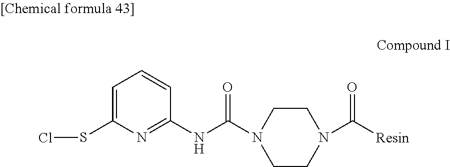

Resin: Polystyrene Resin

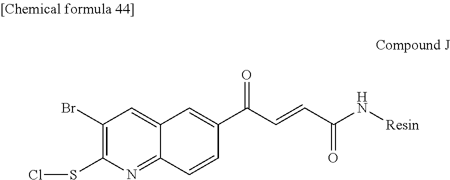

Resin: Polystyrene Resin

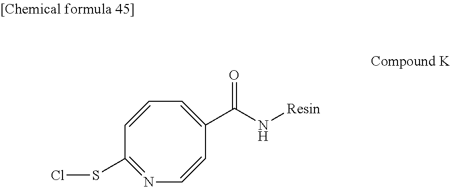

Resin: Polystyrene Resin

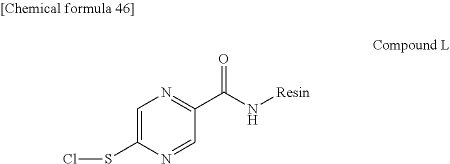

Resin: Polystyrene Resin

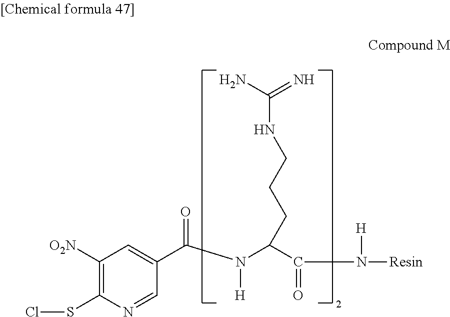

Resin: Polyethylene Glycol-Polystyrene Composite Resin

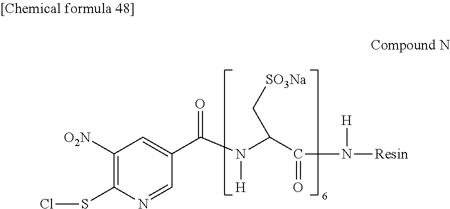

Resin: Polystyrene Resin

Methods for Synthesizing Compounds of the Present Invention

Methods for synthesizing compounds of the present invention are shown below. A method for synthesizing a compound represented by formula (II) wherein the nitrogen-containing heterocycle W in formula (I) is a pyridine ring, L¹ is not present, A^{a} is an amide, A^{b} is not present, and n is 1 is shown first.

Synthesis Scheme of Compound (II)

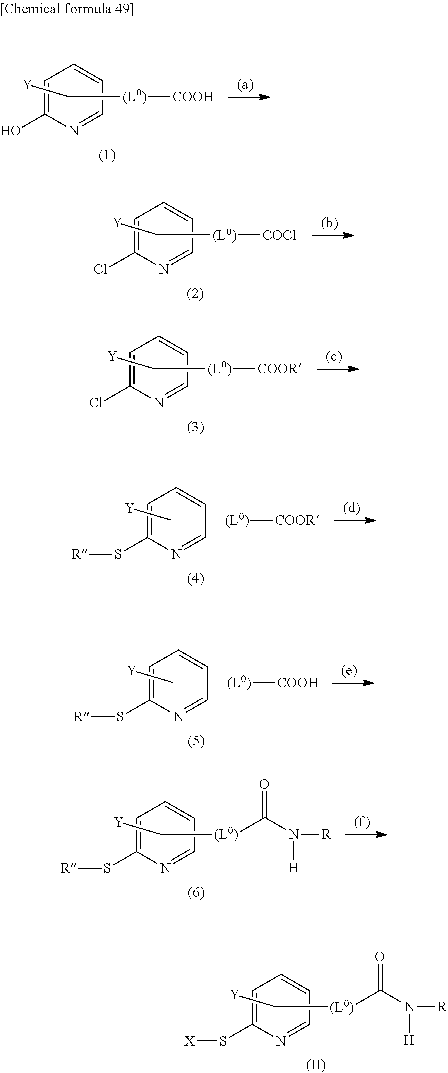

Step (a)

A compound of formula (1) is dissolved in a solvent such as DMF. Thionyl chloride (SOCl₂) is added while cooling the solution by an ice bath or the like in an inert gas stream. The solution is then heated to about 80° C. and reacted for 15-20 hours. The solvent and thionyl chloride are distilled off by concentration, a solvent such as hexane is added, azeotropic distillation is repeated about 3-5 times, and compound (2) is obtained by drying under reduced pressure. Furthermore, a compound of formula (1), for example when Y is a 3-nitro group, can be obtained by reacting fuming nitric acid with 2-hydroxy-5-alkylcarboxy-pyridine.

Step (b)

The compound of formula (2) is reacted with R′OH (wherein R′ represents a C1-C6 alkyl group, for example, a methyl group), and a compound of formula (3) can be synthesized by drying under reduced pressure.

Step (c)

The compound of formula (3) and a primary to tertiary alkylthiol of about C4-25 are dissolved in a solvent such as methanol. A base such as triethylamine is added, and reaction is carried out for several hours under reflux at about 50-70° C. After allowing the reaction solution to cool to room temperature, distilled water is added to the residue obtained by distilling off the solvent under reduced pressure. After extraction by ethyl acetate and drying by anhydrous sodium sulfate or the like, a compound of formula (4) can be synthesized by recrystallizing the solid obtained. In formula (4), R″ is a primary to tertiary carbon serving as a leaving group, for example, benzyl, methoxybenzyl, dimethylaminobenzyl, trityl, chlorotrityl, methyltrityl, methoxytrityl, or t-butyl.

Step (d)

The compound of formula (4) is dissolved in a solvent such as methanol, and the solution is cooled. Lithium hydroxide monohydrate and pure water are then added and reacted for about 20 hours at room temperature. After then distilling off the solvent under reduced pressure, an approximately 10% citric acid aqueous solution is added to the aqueous solution to make a pH of 2-3. The aqueous solution obtained is extracted by ethyl acetate, the solvent is distilled off under reduced pressure, and a compound of formula (5) can be synthesized by drying under vacuum.

Step (e)

The compound of formula (5), an approximately equimolar amount of (O-(7-azabenzotriazol-1-yl)-N,N,N′,N′-tetramethyluronium hexafluorophosphate) (HATU), solvent such as DMF, and diisopropylethylamine are added sequentially to a container and shaken and stirred for 1-2 minutes. Next, this solution is added all at once to a separate container containing H₂N—R (wherein R is a polymeric support used in solid-phase synthesis) and stirred by a magnetic stirrer or by stirring blades, or shaken-stirred by a shaking-stirring solid phase synthesizer (for example, a shaking-stirring solid phase synthesizer KMS-3 manufactured by Kokusan Chemical Co., Ltd.). Stirring is stopped after 1-2 hours, the solvent is filtered out, and about 1 mg of the resin obtained after washing sequentially about 10 times with DMF, about 5 times with methanol, and about 3 times with diethyl ether is taken and subjected to a Kaiser test (free amino group coloring reaction test by a mixed solution of phenol-ethanol solution, potassium cyanide aqueous solution-pyridine solution, and ninhydrin-ethanol solution) to confirm that it is negative. A compound of formula (6) can be synthesized by drying the resin obtained under reduced pressure.

Step (f)

A solvent such as 1,2-dichloroethane is added to the compound of formula (6) and stirred gently for several minutes to swell the solid-phase support. After removing the solvent and cooling, a mixed solution of pyridine, sulfuryl chloride, and 1,2-dichloroethane is added and stirred gently for about 1-2 hours while cooling by ice. After stirring, a small amount of the solution is taken and, after confirming by ¹H-NMR the production of an alkyl product from R″, the solution is removed, and a compound of formula (II) can be synthesized by adding dehydrated dichloromethane and washing several times. Furthermore, chlorine gas, phosphorus oxychloride, phosphorus pentachloride, bromine, fluorinated alkyl pyridine, fluorinated quinuclidine, or iodine can also be used instead of sulfuryl chloride.

Method for Synthesizing a Compound of Formula (II-a)

A compound represented by formula (II-a) wherein the nitrogen-containing heterocycle W in formula (I) is a pyridine ring, L¹ is present, A^{a} is an amide, A^{b} is an amide, and n is 1-5 can be produced by the following synthesis scheme. Furthermore, the compound of formula (II-a) is represented by the formula (II-a′) in the following scheme for the sake of convenience in the description of the synthesis scheme, but both formulas represent the same compounds.

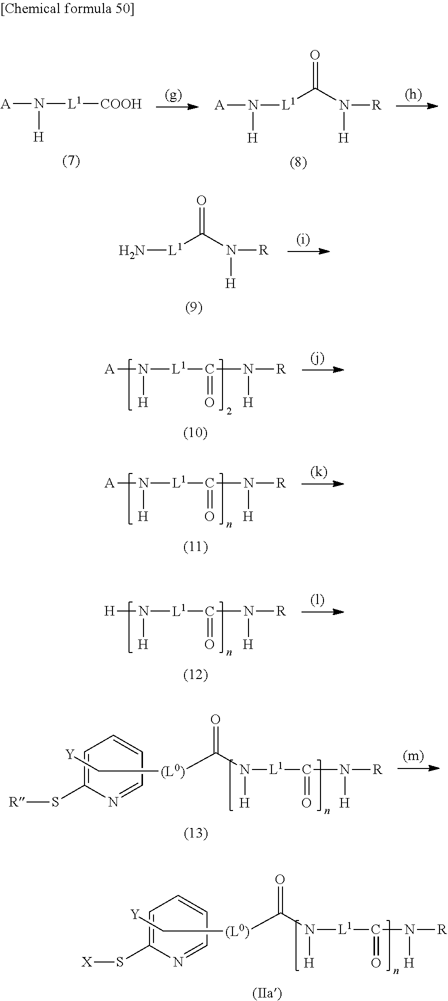

Step (g)

A compound of formula (7), an approximately equimolar amount of a dehydrocondensing agent such as HATU, solvent such as DMF, and diisopropylethylamine are added sequentially to a container and shaken and stirred for 1-2 minutes. In formula (7), A represents an amino group protecting group having a urethane structure, specifically a protecting group of an amino group, and represents a 9-fluorenylmethyloxycarbonyl group, t-butyloxycarbonyl group, benzyloxycarbonyl group, or the like. Next, this solution is added all at once to a separate container containing H₂N—R (wherein R is a polymeric support used in solid-phase synthesis) and shaken and stirred by a shaking-stirring solid-phase synthesizer (for example, a shaking-stirring solid-phase synthesizer KMS-3 manufactured by Kokusan Chemical Co., Ltd.). Stirring is stopped after 1-2 hours, the solvent is filtered out, and about 1 mg of the resin obtained after washing sequentially about 10 times with DMF, about 5 times with methanol, and about 3 times with diethyl ether is taken and subjected to a Kaiser test (amino group coloring reaction test by a mixed solution of phenol-ethanol solution, potassium cyanide aqueous solution-pyridine solution, and ninhydrin-ethanol solution) to confirm that it is negative. A compound of formula (8) can be synthesized by drying the resin obtained under reduced pressure.

Step (h)

A 20% piperidine DMF solution is added to a container containing the compound of formula (8) and shaken and stirred. Stirring is stopped after about 20 minutes, the solvent is filtered out, and the compound of formula (9) obtained by washing about 10 times by dimethylformamide is used as it is in the next reaction. Furthermore, diethylamine, dialkylamine, trifluoroacetic acid, hydrochloric acid, or hydrogen chloride can also be used instead of piperidine.

Step (i)

A compound of formula (7), DMF, and a dehydrocondensing agent (for example, diisopropylcarbodiimide, 1-[bis(dimethylamino)methylene] 1H-benzotriazolium-3-oxide hexafluorophosphate (abbreviation: HBTU), 1-[bis(dimethylamino)methylene] 1H-1,2,3-triazolo(4,5-b)pyridinium 3-oxide hexafluorophosphate (abbreviation: HATU), bromotris(pyrrolidino)phosphonium hexafluorophosphate (abbreviation: PyBrop) hydroxybenzotriazole hydrate) are added sequentially to a container containing the compound of formula (9) and shaken and stirred. Stirring is stopped after 1-2 hours, the solvent is filtered out, and a compound of formula (10) can be obtained by washing about 10 times with dimethylformamide. This compound is used as it is in the next reaction. About 1 mg of the compound obtained is taken separately and subjected to a Kaiser test (amino group coloring reaction test by a mixed solution of phenol-ethanol solution, potassium cyanide aqueous solution-pyridine solution, and ninhydrin-ethanol solution) to confirm that it is negative.

Step (j)

A compound of formula (11) is obtained by alternately repeating the above step (h) and step (i) n-2 times using the compound of formula (10). The compound of formula (11) obtained is used as it is in the next reaction. Furthermore, this step is not necessary when a compound in which n is 1 in formula (II-a) is obtained, and the compound of formula (10) is supplied to step (k).

Step (k)

A 20% piperidine DMF solution is added to a container containing the compound of formula (11) and shaken and stirred. Stirring is stopped after about 20 minutes, the solvent is filtered out, and a compound of formula (12) is obtained by washing about 10 times by dimethylformamide and used as it is in the next reaction. Furthermore, diethylamine, dialkylamine, trifluoroacetic acid, hydrochloric acid, or hydrogen chloride can also be used suitably in accordance with the type of A instead of piperidine.

Step (l)

A compound of formula (5), an approximately equimolar amount of HATU, DMF, and an approximately equimolar amount of diisopropylethylamine are added sequentially to a container and shaken and stirred for about one minute. This solution is added all at once to a container containing the compound of formula (12) and shaken and stirred. Stirring is stopped after 1-2 hours, the solvent is filtered out, and a compound of formula (13) can be synthesized by drying under reduced pressure after washing sequentially about 10 times with dimethylformamide, about 5 times with methanol, and about 3 times with diethyl ether. About 1 mg of the compound obtained is taken separately and subjected to a Kaiser test to confirm that it is negative.

Step (m)

A solvent such as 1,2-dichloroethane is added to the compound of formula (13) and stirred gently for several minutes to swell the solid-phase support. After removing the solvent and cooling, a mixed solution of pyridine, sulfuryl chloride, and 1,2-dichloroethane is added and stirred gently for about 1-2 hours while cooling by ice. After stirring, a small amount of the solution is taken and, after confirming by ¹H-NMR the production of an alkyl product from R″, the solution is removed. A compound of formula (II-a′) can be synthesized by adding dehydrated dichloromethane and washing several times.

SH Group Selective Reactive Solid-Phase Supported Reagent of the Present Invention

Compounds of the present invention can be used as solid-phase supported reagents that react selectively with compounds having SH groups since they can be immobilized on a polymeric support used in solid-phase synthesis. Specifically, one embodiment of the present invention is an SH group selective reactive solid-phase supported reagent containing a compound of formula (I), (II), or (II-a). Here, SH group selective reactivity means bonding by reacting selectively with the SH groups of a compound having SH groups.

Another embodiment of the present invention is a method for introducing S—S bonds by reacting a compound of formula (I), (II), or (II-a) with an organic compound having SH groups or an organic compound in which the SH groups are protected by protecting groups. Specifically, one embodiment of the present invention is a method for producing a compound represented by formula (IV) by reacting a compound represented by formula (I) with a compound represented by formula (III) (also called “production method 1 of the present invention” hereinafter).

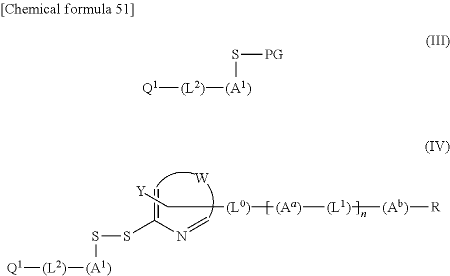

In formulas (III) and (IV), Q¹ represents an organic compound. Q¹ is selected from the group consisting of biological organic compounds, selected from amino acids, peptides, proteins, antibodies, nucleic acid bases, nucleotides or nucleosides, polymer compounds, low-molecular compounds, fluorescent labeling substances, enzyme labeling substances, biotin, chelating agents, and derivatives thereof including isotopes of these.

Essential amino acids, β-alanine and other such β-amino acids, γ-aminobutyric acid and other such γ-amino acids, stable isotope-modified amino acids including deuterated amino acids, and the like can be used as amino acids. A¹ when present, L¹ when present, and S-PG may bond to either the main chain or side chain of the amino acid.

Examples of peptides includes various oligopeptides, for example, oligoarginine, polylysine, cell adhesion factor peptides such as arginyl-glycyl-asparagine, cell death-inducing peptides such as lysyl-leucyl-alanyl-lysine, and the like.

Examples of proteins include laminin, CFP, GFP, YFP, allophycocyanin, phycoerythrin, and the like.

Examples of antibodies include monoclonal antibodies and the like.

Examples of nucleic acid bases, nucleotides or nucleosides include adenine, guanine, thymine, uracil, cytosine, AMP, ADP, ATP, GTP, UTP, CTP, their derivatives including deoxynucleotide dATP, and the like.

Examples of polymer compounds include synthetic rubber, synthetic resins, synthetic fibers, natural rubber, starch, sugar chains, fats and oils, and the like.

Examples of low-molecular compounds include sialic acid, cholesterol, vitamins, alkaloids, steroids, cyclodextrin, crown ethers, EDTA, and the like and radioactive isotopes and stable isotopes of these, and the like.

Examples of fluorescent labeling substances include fluorescein, coumarin, eosin, phenanthroline, pyrene, rhodamine, indocyanine, quinoxaline, derivatives of these, and the like, for example, substances derived from fluorescein isothiocyanate.

Examples of enzyme labeling substances include β-galactosidase, alkaline phosphatase, glucose oxidase, peroxidase, and the like.

Q¹ may also be two or more organic compounds selected from the group consisting of biological organic compounds, selected from amino acids, peptides, proteins, antibodies, nucleic acid bases, nucleotides or nucleosides, polymer compounds, low-molecular compounds, fluorescent labeling substances, enzyme labeling substances, biotin, chelating agents, and their derivatives including isotopes of these in bonded form. When two or more organic compounds are bonded, they may be bonded via a linker (straight or branched C1-C10 alkylene, C2-C10 alkenylene, C2-C10 alkynylene, cycloalkylene having 3-10 carbon atoms, cycloalkenylene having 3-10 carbon atoms, arylene, monocyclic heteroarylene, heterocycle, amine, amide, ether, ester, sulfide, carboxylic acid, sulfonic acid, sulfonamide, ketone, polyethylene glycol chain, polyamide, and the like).

In formulas (III) and (IV), L², when present, represents a linker having a chemically stable structure. Linkers represented as L² are selected from the group consisting of straight or branched C1-C10 alkylenes, C2-C10 alkenylenes, C2-C10 alkynylenes, cycloalkylenes having 3-10 carbon atoms, cycloalkenylenes having 3-10 carbon atoms, arylenes, monocyclic heteroarylenes, heterocycles, amines, amides, ethers, esters, sulfides, carboxylic acids, sulfonic acids, sulfonamides, ketones, polyethylene glycol chains, polyamides, and groups represented by formula (a)

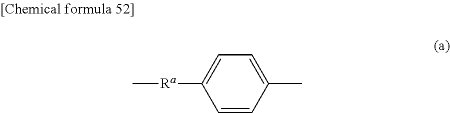

(in the formula, R^{a} represents an optionally substituted C1-C15 alkylene. Here, arbitrary substituents can be selected as substituents; examples include alkyl groups, optionally substituted (for example, alkyl groups, alkoxy groups, and the like) aryl groups, alkoxy groups, and the like); these alkylenes, alkenylenes, alkynylenes, cycloalkylenes, cycloalkenylenes, arylenes, and monocyclic heteroarylenes are optionally substituted, and arbitrary substituents can be selected as substituents. Preferred as L² are C2-C6 alkylenes, polyethylene glycol having a molecular weight of 100-1000, and polyamides.

In formulas (III) and (IV), A¹, when present, represents a functional group having S-PG. A¹ may not be present; in this case, S-PG may bond directly to a linker or may bond directly to an organic compound of Q¹.

Examples of A¹ with S-PG bonded, that is, A¹-S-PG, include cysteine, cysteine in which the SH group is protected by a protecting group, cysteinamide, cysteinamide in which the SH groups are protected by protecting groups, cysteamine, cysteamine in which the SH group is protected by a protecting group, acetylcysteine, acetylcysteine in which the SH group is protected by a protecting group, aminoalkylthiol, aminoalkylthiol in which the SH group is protected by a protecting group, mercaptoethanol, and mercaptoethanol in which the SH group is protected by a protecting group.

In formula (III), PG represents an SH group protecting group or a hydrogen atom. SH group protecting groups are selected from t-butyl, trityl, benzhydryl, benzyl, methylbenzyl, dimethylbenzyl, trimethylbenzyl, methoxybenzyl, dimethoxybenzyl, trimethoxybenzyl, nitrobenzyl, acetamidomethyl, 9-fluorenylmethyl, carbonylbenzyloxy, diphenylbenzyl, ethylcarbamoyl, picolyl, sulfonyl, or salts thereof.

One aspect of the present invention is a method for producing a compound represented by formula (IVa) by reacting a compound represented by formula (II) with a compound represented by formula (III) (also called “production method 1a of the present invention” hereinafter).

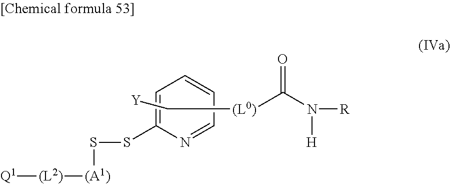

L⁰ and R are as defined in formula (II), and Q¹, L², and A¹ are as defined in formula (III).

Another aspect of the present invention is a method for producing a compound represented by formula (IVb) by reacting a compound represented by formula (II-a) with a compound represented by formula (III) (also called “production method 1b of the present invention” hereinafter).

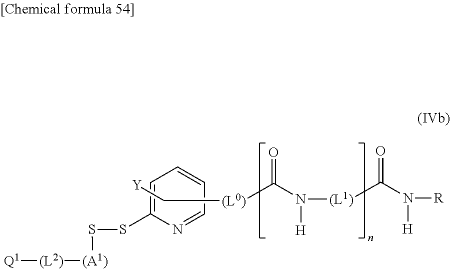

L⁰, L⁰, R, and n are as defined in formula (II-a), Q¹, L², and A¹ are as defined in formula (III).

The production method 1, 1a, or 1b of the present invention can be carried out by the following procedure.

A compound of formula (I), (II), or (II-a) is placed in a container, and a solution of 1.2-50 equivalents, more preferably 20-50 equivalents, relative to the functional group substitution rate on the solid-phase support, of a compound of formula (III) are added. After 2-8 hours, the solution is removed by filtration, and a compound of formula (IV), (IVa), or (IVb) can be obtained by washing 10 times using the solvent used, as well as 5 times by methanol, and 5 times by diethyl ether, and drying under reduced pressure. Furthermore, an organic solvent or aqueous solution can be selected as is convenient as the solvent used as long as it is a solvent capable of adequately dissolving the compound of formula (III). Examples include dichloromethane, dichloroethane, dimethylformamide, acetonitrile, ethyl acetate, methanol, hexane, diethyl ether, tetrahydrofuran, trifluoroacetic acid, trifluoroethanol, distilled water, buffers, acetic acid, hydrochloric acid, and formic acid; dichloromethane, distilled water, formic acid, acetic acid, and trifluoroacetic acid are preferred.

Nonlimiting examples of compounds that can be produced using the production method 1, 1a, or 1b of the present invention appear below.

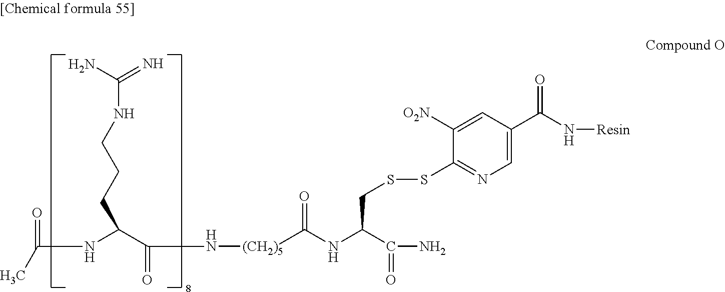

Resin: Crosslinked Polyethylene Glycol (Methyl ChemMatrix® Resin)

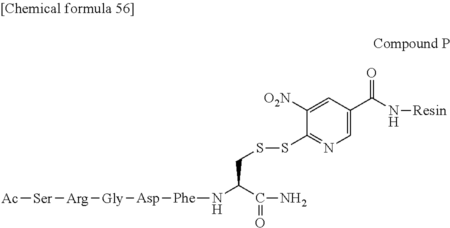

Resin: Crosslinked Polyethylene Glycol (Methyl ChemMatrix® Resin)

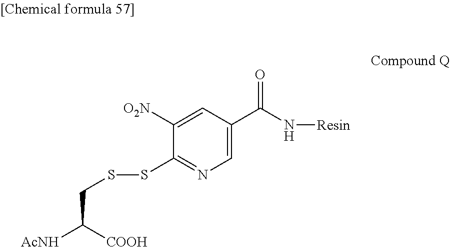

Resin: Crosslinked Polyethylene Glycol (Methyl ChemMatrix® Resin)

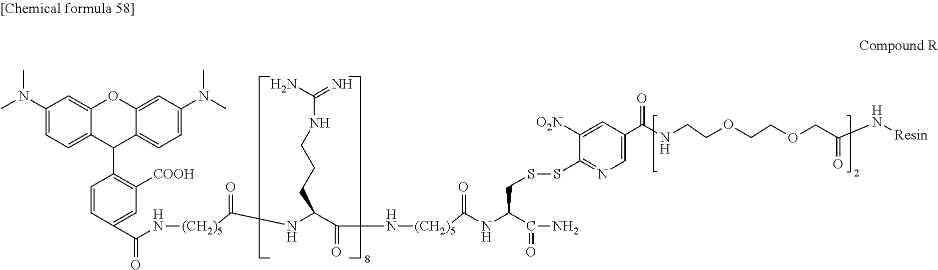

Resin: Polyethylene Glycol-Polystyrene Composite Resin

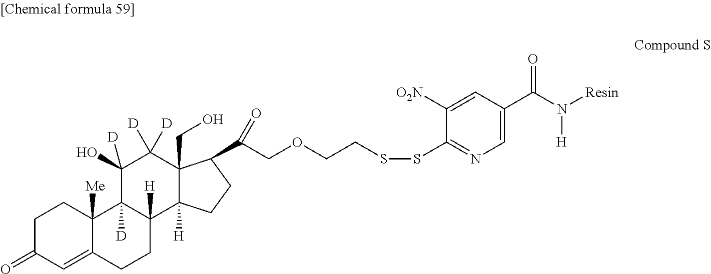

Resin: Polystyrene Composite Resin

Another embodiment of the present invention is a method for producing a compound having S—S bonds by reacting a compound of formula (IV) with another organic compound having SH groups or an organic compound having SH groups protected by protecting groups. Specifically, one embodiment of the present invention is a method for producing a compound represented by formula (VI) by reacting a compound represented by formula (IV) with a compound represented by formula (V) (also called “production method 2 of the present invention” hereinafter).

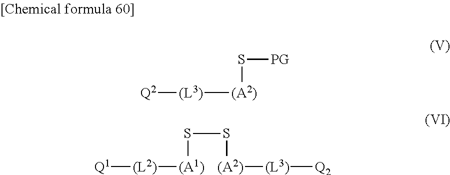

In formulas (V) and (VI), Q² represents an organic compound as in Q¹, and Q² is selected from the group consisting of biological organic compounds, selected from amino acids, peptides, proteins, antibodies, nucleic acid bases, nucleotides or nucleosides, polymer compounds, low-molecular compounds, fluorescent labeling substances, enzyme labeling substances, biotin, chelating agents, and derivatives thereof including isotopes of these. Organic compounds that can be used as Q² are the same as those given as examples of Q¹.

Q² may also be two or more organic compounds selected from the group consisting of biological organic compounds, selected from amino acids, peptides, proteins, antibodies, nucleic acid bases, nucleotides or nucleosides, polymer compounds, low-molecular compounds, fluorescent labeling substances, enzyme labeling substances, biotin, chelating agents, and derivatives thereof including isotopes of these in bonded form. When two or more organic compounds are bonded, they may be bonded via a linker given as an example for Q¹.

In formulas (V) and (VI), L³, when present, represents a linker having a chemically stable structure. Linkers represented as L³ are selected from the group consisting of straight or branched C1-C10 alkylenes, C2-C10 alkenylenes, C2-C10 alkynylenes, cycloalkylenes having 3-10 carbon atoms, cycloalkenylenes having 3-10 carbon atoms, arylenes, monocyclic heteroarylenes, heterocycles, amines, amides, ethers, esters, sulfides, ketones, polyethylene glycol chains, polyamides, and groups represented by formula (a)

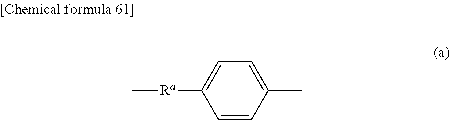

(in the formula, R^{a} represents an optionally substituted C1-C15 alkylene. Here, arbitrary substituents can be selected as substituents; examples include alkyl groups, optionally substituted (for example, alkyl groups, alkoxy groups, and the like) aryl groups, alkoxy groups, and the like.); these alkylenes, alkenylenes, alkynylenes, cycloalkylenes, cycloalkenylenes, arylenes, and monocyclic heteroarylenes are optionally substituted, and arbitrary substituents can be selected as substituents. Preferred as L³ are C2-C6 alkylenes, polyethylene glycol having a molecular weight of 100-1000, and polyamides.

In formulas (V) and (VI), A², when present, represents a functional group having S-PG. A² may not be present; in this case, S-PG may bond directly to a linker or may bond directly to an organic compound of Q².

Examples of A² with S-PG bonded, that is, A²-S-PG, include cysteine, cysteine in which the SH group is protected by a protecting group, cysteinamide, cysteinamide in which the SH group is protected by a protecting group, cysteamine, cysteamine in which the SH group is protected by a protecting group, acetylcysteine, acetylcysteine in which the SH group is protected by a protecting group, aminoalkylthiol, aminoalkylthiol in which the SH group is protected by a protecting group, mercaptoethanol, and mercaptoethanol in which the SH group is protected by a protecting group. Preferably, A² is not present, and the SH group protecting group PG is a hydrogen atom or methoxytrityl group; in other words, it has a structure of Q²-L³-S-PG in which L³ and PG are directly bonded. More preferably, PG is a hydrogen atom, preferably having a structure of Q²-L³-S—H.

In formula (V), PG represents an SH group protecting group or a hydrogen atom. SH group protecting groups are selected from t-butyl, trityl, benzhydryl, benzyl, methylbenzyl, dimethylbenzyl, trimethylbenzyl, methoxybenzyl, dimethoxybenzyl, trimethoxybenzyl, nitrobenzyl, acetamidomethyl, 9-fluorenylmethyl, carbonylbenzyloxy, diphenylbenzyl, ethylcarbamoyl, picolyl, sulfonyl, or salts thereof.

One aspect of the present invention is a method for producing a compound represented by formula (VI) by reacting a compound represented by formula (IVa) with a compound represented by formula (V) (also called “production method of the present invention 2a” hereinafter).

Another aspect of the present invention is a method for producing a compound represented by formula (VI) by reacting a compound represented by formula (IVb) with a compound represented by formula (V) (also called “production method 2b of the present invention” hereinafter).

The production method 2, 2a, or 2b of the present invention can be carried out by the following procedure.

(1) A compound of formula (V) is dissolved in a solvent. According to a preferred embodiment, a compound of formula (V) is dissolved in water or an organic solvent containing 1% or more of water. The pH at this time is preferably close to neutral, preferably pH 6.5-8.5. A buffer can also be used instead of water, and water, buffer, and organic solvent can be used in any combination. On the other hand, when an organic solvent is used in combination, the organic solvent is preferably miscible with water. Examples include acetonitrile, dimethylformamide, acetone, dimethylsulfoxide, alcohol, tetrahydrofuran, 1,4-dioxane, and the like.

(2) The solution of the compound of formula (V) prepared above in (1) is mixed with a compound of formula (IV), (IVa), or (IVb). A compound of formula (IV) or (IVa) may be added to a container containing the solution at this time, or the solution may be added to a container containing a compound of formula (IV), (IVa), or (IVb). Furthermore, the form and material of which the container is made are not restricted, but a container that permits stirring equipped with a filter for filtration such as a tube with a filter is preferred. The container may be allowed to stand for mixing, but it is preferable to conduct shaking or stirring by a shaker for solid-phase synthesis, magnetic stirrer, vortex mixer, three-one motor, or the like.

(3) A reaction can usually be carried out for from 5 minutes to two hours by the reaction that occurs due to mixing in (2) above. The amount of compound of formula (IV), (IVa), or (IVb) used in this reaction may be increased or decreased in accordance with the amount of compound of formula (V). For example, it is preferable to use an excess as the amount of compound of formula (IV), (IVa), or (IVb) per equivalent of compound of formula (V), more preferably from 1.2 to 10 equivalents. Completion of the reaction can be determined by determining the consumption of the compound of formula (V) in the solution by a common analytical method. Examples of applicable analytical methods appropriately include HPLC, NMR, TLC, IR, MS spectrum, titration, and the like, and a method suited to the detection of formulas (V) and (IV) can be appropriately utilized.

(4) After the reaction, the compound of formula (VI), unreacted compound of formula (I), (II), or (IIa), and compounds into which formulas (I), (II), or (IIa) change as the reaction progresses are separated by filtration, and a compound of formula (VI) is obtained as a solution in the filtrate. The instrument and method used in filtration are not restricted. Examples of instruments include filter paper, glass fibers, filtration adjuvants, filtration by filter cloth, and membrane filter, glass filter, and the like. Examples of the filtration technique include natural filtration, suction filtration, centrifugal separation, decantation, and the like, and can be selected as is appropriate in accordance with the use and reaction scale.

Nonlimiting examples of compounds that can be produced using the production method 2, 2a, or 2b of the present invention appear below.

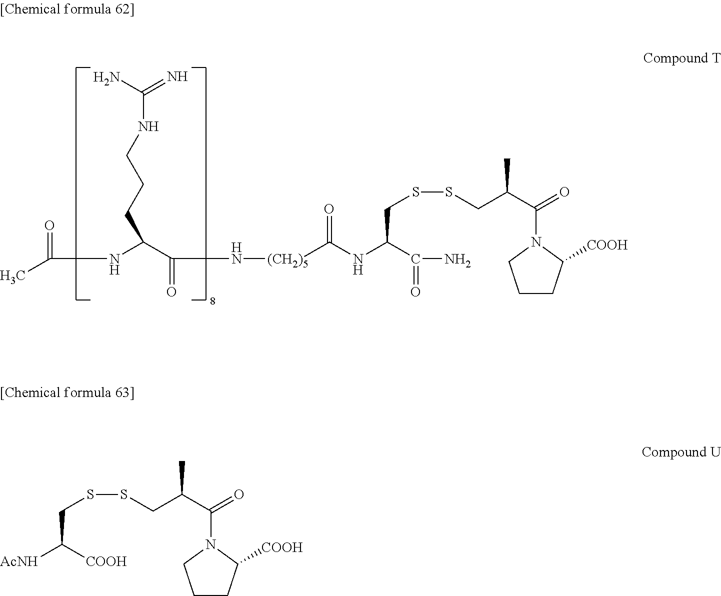

Compounds T and U are compounds obtained by asymmetric disulfide synthesis by reacting compounds O and Q, respectively, with captopril.

Yet another embodiment of the present invention is a method for producing a compound represented by formula (IV) by reacting a compound represented by formula (I) with a compound represented by formula (III), and producing a compound represented by formula (VI) by reacting the compound represented by formula (IV) with a compound represented by formula (V).

Yet another aspect of the present invention is a method for producing a compound represented by formula (IVa) by reacting a compound represented by formula (II) with a compound represented by formula (III), and producing a compound represented by formula (VI) by reacting the compound represented by formula (IVa) with a compound represented by formula (V).

Yet another aspect of the present invention is a method for producing a compound represented by formula (IVb) by reacting a compound represented by formula (IIa) with a compound represented by formula (III), and producing a compound represented by formula (VI) by reacting the compound represented by formula (IVb) with a compound represented by formula (V).

Yet another embodiment of the present invention is a method for producing a compound having S—S bonds by reacting a compound of formula (IV) with an organic compound having two SH groups in which one SH group is protected by a protecting group. Specifically, one embodiment of the present invention is a method for producing a compound represented by formula (VIa) by reacting a compound represented by formula (IV) with a compound represented by formula (Va) (also called “production method 3 of the present invention” hereinafter).

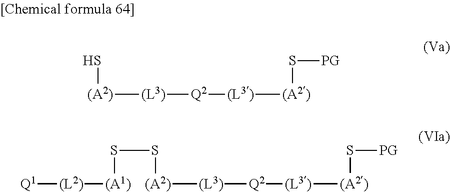

(wherein L^{3′} and A^{2′} are defined in the same way as L³ and A², respectively.)

Nonlimiting examples of compounds that can be produced using the production method 3 of the present invention appear below.

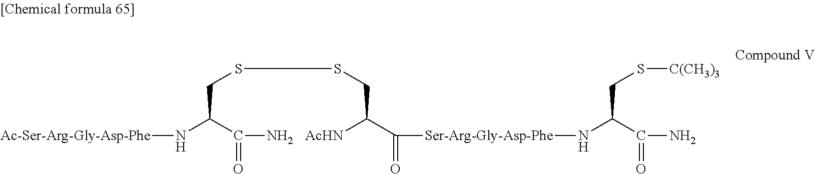

Compound V is a compound obtained by reacting compound P and H-Cys-Ser-Arg-Gly-Asp-Phe-Cys(tBu)-NH₂.

A compound of formula (VIa) can also be reacted with a compound of formula (I), and the compound obtained reacted with a compound represented by formula (Va) or formula (V). Repeating this reaction makes it possible to obtain a compound in which fragments of Q are connected as shown below.

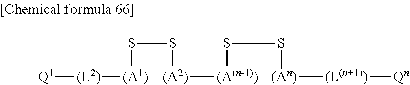

(wherein L⁽ⁿ⁺¹⁾ is defined in the same way as L², A⁽ⁿ⁺¹⁾ and Aⁿ are defined in the same way as A¹, and Qⁿ is defined in the same way as Q¹.)

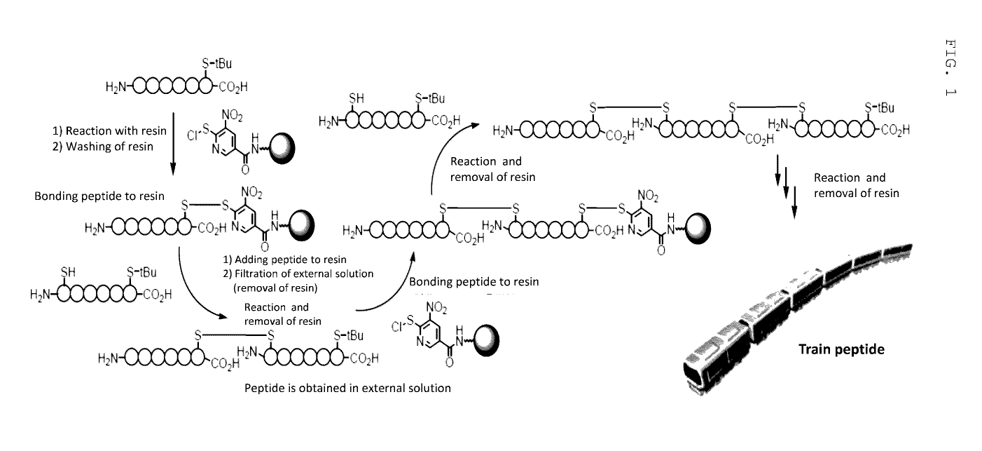

Thus, the use of a compound of formula (I) makes it possible, for example, to produce a compound having many peptide fragments connected (train peptide) when Q is a peptide. FIG. 1 shows a scheme for synthesizing a train peptide. As shown in FIG. 1, the use of a compound of formula (I) makes it possible to connect peptide fragments even without protecting the peptide ends.

In addition, S—S bonds are finally formed after all of the peptide bonds have been connected in conventional peptide synthesis. However, since peptide fragments are connected by S—S bonds from the start, meaning that specific peptide bonds are formed by intramolecular reaction, this makes it possible to provide a new peptide synthesis technique.

### EXAMPLES

The present invention is explained below through examples, but these do not limit the scope of the present invention.

### Example 1

The synthesis of compound A is shown below as an example of a compound of the present invention.

### Synthesis of compound A (6-chlorosulfenyl-5-nitronicotinemethylamide resin)

Compound A was synthesized according to the following scheme.

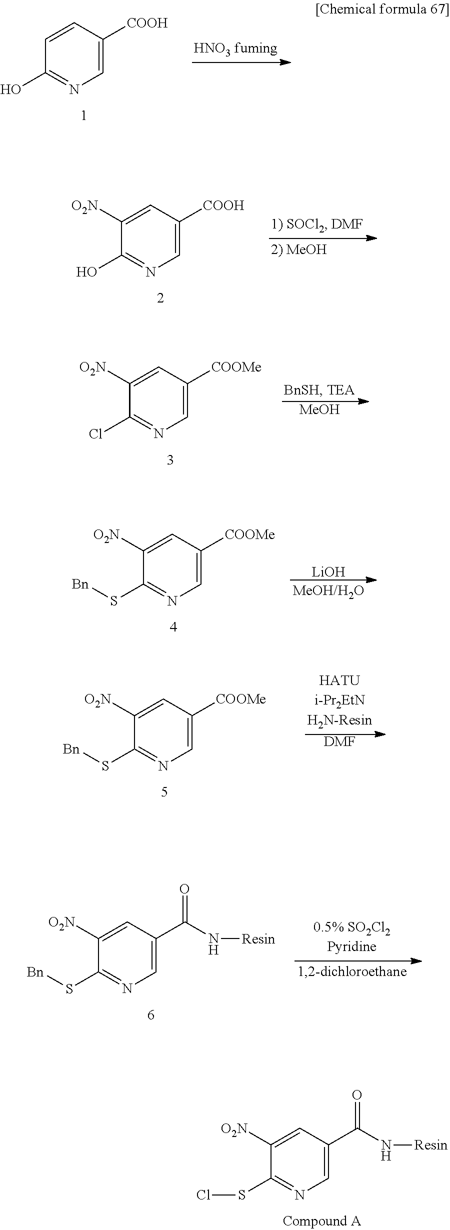

Resin: Crosslinked Polyethylene Glycol (Methyl ChemMatrix® Resin)

(1) Synthesis of Compound 2

Compound 1 (25 g, 0.180 mol) was placed in a 500 mL recovery flask, and fuming nitric acid (1.52) (125 mL) was added. The flask was gradually heated using an oil bath while stirring, and stirring was conducted for 5 hours at 50° C. After stopping heating and allowing to cool to room temperature, the reaction solution was concentrated under reduced pressure. The residue obtained was cooled by an ice bath, and recrystallized using methanol as the solvent. Compound 2 (9.77 g, 0.053 mol) was obtained by drying the solid obtained by filtration, under reduced pressure.

1H NMR (300 MHz, CD₃OD) 8.44 (d, J=2.6 Hz, 1H), 8.85 (d, J=2.6 Hz, 1H); HRMS (ES+): m/z 185.0194 (M+H)⁺ (calcd for C₆H₆N₂O₆: 185.0198).

(2) Synthesis of Compound 3

Compound 2 (20.0 g) and N,N-dimethylformamide (8.44 mL, 0.109 mmol) were added to a 500 mL recovery flask in an argon gas stream, and thionyl chloride (158.3 mL, 2.18 mmol) was added while cooling by ice bath. After the entire amount of thionyl chloride had been added, the flask was returned to room temperature. It was then gradually heated using an oil bath, and stirring was conducted for 16 hours at 80° C. After stopping heating and allowing to cool to room temperature, the reaction solution was concentrated under reduced pressure. Dichloromethane (50 mL) was added to the residue obtained, which was again concentrated, and the remaining thionyl chloride was distilled off azeotropically. After concentration, the residue obtained was cooled by an ice bath, and methanol (50 mL) was added. Compound 3 (18.2 g, 0.084 mmol) was obtained by concentration and drying under reduced pressure.

1H NMR (300 MHz, CD₃OD) 4.00 (s, 3H), 8.52 (d, J=2.1 Hz, 1H), 9.14 (d, J=2.1 Hz, 1H); HRMS (ES+): m/z 217.0006 (M+H)⁺ (calcd for C₇H₈N₂O₄Cl: 217.0016).

(3) Synthesis of Compound 4

Methanol (15 mL) was placed in a 100 mL recovery flask, and compound 3 (2.54 g, 11.7 mmol) and benzyl mercaptan (2.18 g, 17.6 mmol) were added and dissolved while stirring. After confirming that the raw materials were completely dissolved, triethylamine (2.46 mL) was added. The external temperature was set at 60° C. using an oil bath, and stirring was carried out for 5 hours under reflux. After allowing the reaction solution to cool to room temperature, the solvent was distilled off under reduced pressure, distilled water was added to the residue obtained, and extraction was performed by ethyl acetate. The organic phase was washed with distilled water and saturated saline, respectively, and dried by anhydrous sodium sulfate. After filtering out the anhydrous sodium sulfate, the solvent was distilled off under reduced pressure, the solid obtained was recrystallized from hexane and ethyl acetate, and compound 4 (3.27 g, 10.7 mmol) was obtained.

1H NMR (300 MHz, CDCl₃) 4.00 (s, 3H), 4.52 (s, 2H), 7.18-7.36 (m, 3H), 7.38-7.48 (m, 2H), 9.02 (d, J=1.9 Hz, 1H), 9.25 (d, J=1.8 Hz, 1H); HRMS (ES+): m/z 305.0592 (M+H)⁺ (calcd for C₁₄H₁₃N₂O₄S: 305.0596).

(4) Synthesis of Compound 5

Compound 4 (3.27 g, 10.7 mmol) was placed in a 500 mL recovery flask, methanol (210 mL) was added, and the flask was cooled using an ice bath. Next, lithium hydroxide monohydrate (902 mg, 21.5 mmol) and pure water (180 mL) were added. After stirring for 10 minutes in the ice bath, the temperature was raised to room temperature, and stirring was conducted for 15 hours. A solution obtained by dissolving lithium hydroxide monohydrate (225 mg, 5.4 mmol) in pure water (5 mL) was also added and stirred for 4.5 hours. The solution was confirmed to have become clear, and the methanol was distilled off under reduced pressure. 10% Citric acid aqueous solution was added to the remaining aqueous solution until the pH reached 3. The aqueous solution obtained was extracted using ethyl acetate, and the organic phase was washed sequentially by water and saturated saline, then dried using anhydrous sodium sulfate. The anhydrous sodium sulfate was filtered out, the solvent was distilled off under reduced pressure, and compound 5 (3.1 g, 10.7 mmol) was obtained by drying under vacuum.

1H NMR (300 MHz, CD₃OD) 4.52 (s, 2H), 7.18-7.36 (m, 3H), 7.38-7.48 (m, 2H), 8.94 (d, J=2.0 Hz, 1H), 9.22 (d, J=2.0 Hz, 1H); HRMS (ES+): m/z 291.0442 (M+H)⁺ (calcd for C₁₃H₁₁N₂O₄S: 291.0440).

(5) Synthesis of Compound 6

Compound 5 (508.1 mg, 1.75 mmol), (O-(7-azabenzotriazol-1-yl)-N,N,N′,N′-tetramethyluronium hexafluorophosphate (528.2 mg, 1.72 mmol), DMF (16 mL), and diisopropylethylamine (251.0 μL) were added sequentially to a 15 mL polypropylene tube and shaken and stirred for one minute. Five hundred milligrams of aminomethyl ChemMatrix resin (in the formula, H₂N-Resin, functional group substitution rate 0.70 mmol/g) was placed in another 60 mL polypropylene tube equipped with a filtration frit, and the solution in the 15 mL tube was added to it all at once. The tube was installed in a shaking-stirring solid-phase synthesizer KMS-3 (manufactured by Kokusan Chemical Co., Ltd.) and shaken and stirred. Stirring was stopped after 1.5 hours, the solvent was filtered out, and the product was washed 10 times with 5 mL of dimethylformamide, 5 times with methanol, and 3 times with diethyl ether, sequentially. One milligram of the resin obtained was taken and confirmed to be negative when subjected to a Kaiser test (free amino group coloring reaction test by a mixed solution of phenol-ethanol solution, potassium cyanide aqueous solution-pyridine solution, and ninhydrin-ethanol solution). Compound 6 (560 mg) was obtained by drying the compound obtained under reduced pressure.

(6) Synthesis of Compound A

A stirring bar and the resin compound (30.7 mg) were placed in a 10 mL glass test tube. 1,2-Dichloroethane (2.0 mL) was added and stirred gently to swell the resin. After stirring for 5 minutes, the solvent was removed by a Pasteur pipette. The test tube was then cooled using an ice bath, and a mixed solution of pyridine (7.3 μL), sulfuryl chloride (10 μL), and 1,2-dichloroethane (1.99 mL) prepared in a separate 30 mL Erlenmeyer flask was added and stirred gently while cooling by ice. After stirring for 1.5 hours, the solution was removed using a Pasteur pipette, dehydrated dichloromethane (2 mL) was added, and the resin compound was washed. After removing the wash solution by Pasteur pipette, dichloromethane was again added, and compound A was obtained by washing in the same way 5 times.

### Example 2

The synthesis of compound B is shown below as an example of a compound of the present invention.

### Synthesis of compound B (5-((6-(methylamino resin)-6-oxohexyl)amino)-6-oxohexyl)carbonyl)-3-nitropyridine-2-sulphenyl chloride)

Compound B was synthesized according to the following scheme.

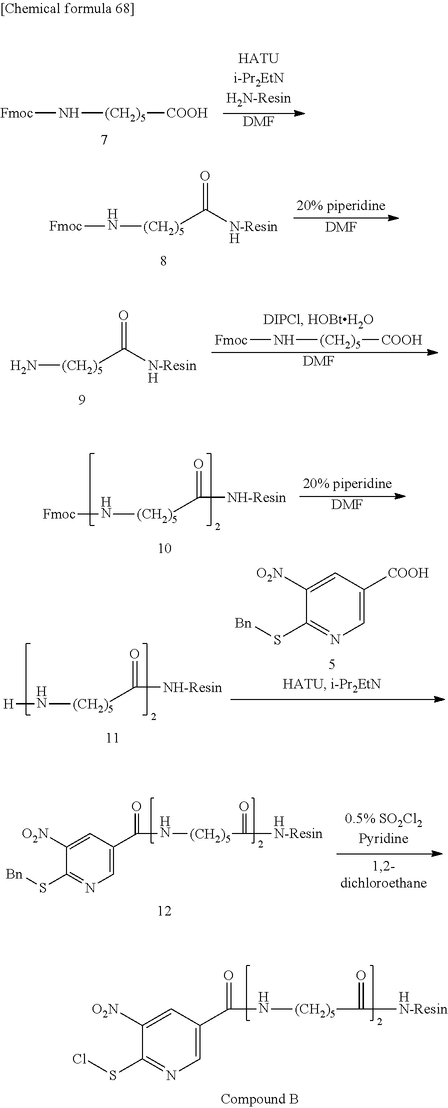

Resin: Crosslinked Polyethylene Glycol (Methyl ChemMatrix® Resin)

(1) Synthesis of Compound 8

9-Fluorenylmethyloxycarbonylaminocaproic acid (compound 7, 632.4 mg, 1.79 mmol), (O-(7-azabenzotriazol-1-yl)-N,N,N′,N′-tetramethyluronium hexafluorophosphate) (540.6 mg, 1.76 mmol), DMF (16 mL), and diisopropylethylamine (257.0 μL, 1.79 mmol) were added sequentially to a 15 mL polypropylene tube and shaken and stirred for one minute. A quantity of 511.7 mg of aminomethyl ChemMatrix resin (in the formula, H₂N-Resin, functional group substitution rate 0.70 mmol/g) was placed in a separate 60 mL polypropylene tube equipped with a filtration frit, and the solution in the 15 mL tube was added to it all at once. The tube was installed in a shaking-stirring solid-phase synthesizer KMS-3 (manufactured by Kokusan Chemical Co., Ltd.) and shaken and stirred. Stirring was stopped after 1.5 hours, the solvent was filtered out, and a resin compound 8 was obtained by washing 10 times with 5 mL of dimethylformamide and used as it was in the next reaction. One milligram of the resin obtained was taken separately and confirmed to be negative when subjected to a Kaiser test (free amino group coloring reaction test by a mixed solution of phenol-ethanol solution, potassium cyanide aqueous solution-pyridine solution, and ninhydrin-ethanol solution).

(2) Synthesis of Compound 9

Sixteen milliliters of 20% piperidine DMF solution was added to a 60 mL polypropylene tube equipped with a filtration frit containing compound 8 and shaken and stirred. Stirring was stopped after 20 minutes, the solvent was filtered out, and resin compound 9 was obtained by washing 10 times by 5 mL of dimethylformamide and used as it was in the next reaction.

(3) Synthesis of Compound 10

9-Fluorenylmethyloxycarbonylamincaproic acid (compound 7, 632.4 mg, 1.79 mmol), DMF (16 mL), diisopropylcarbodiimide (201.0 mg, 1.79 mmol), and hydroxybenzotriazole hydrate (274.2 mg, 1.79 mmol) were added sequentially to a 60 mL polypropylene tube equipped with a filtration frit containing compound 9 and shaken and stirred. Stirring was stopped after 1.5 hours, the solvent was filtered out, and compound 10 was obtained by washing 10 times by 5 mL of dimethylformamide and used as it was in the next reaction. One milligram of the resin obtained was taken separately and confirmed to be negative when subjected to a Kaiser test (free amino group coloring reaction test by a mixed solution of phenol-ethanol solution, potassium cyanide aqueous solution-pyridine solution, and ninhydrin-ethanol solution).

(4) Synthesis of Compound 11

Sixteen milliliters of 20% piperidine DMF solution was added to a 60 mL polypropylene tube equipped with a filtration frit containing compound 10 and shaken and stirred. Stirring was stopped after 20 minutes, the solvent was filtered out, and compound 11 was obtained by washing 10 times by 5 mL of dimethylformamide and used as it was in the next reaction.

(5) Synthesis of Compound 12

Compound 5 (519.9 mg, 1.79 mmol), (O-(7-azabenzotriazol-1-yl)-N,N,N′,N′-tetramethyluronium hexafluorophosphate) (540.6 mg, 1.76 mmol), DMF (16 mL), and diisopropylethylamine (257.0 μL, 1.79 mmol) were added sequentially to a 15 mL polypropylene tube and shaken and stirred for one minute. This solution was added all at once to a 60 mL polypropylene tube equipped with a filtration frit containing compound 11 and shaken and stirred. Stirring was stopped after 1.5 hours, the solvent was filtered out, and compound 12 (623.4 mg) was obtained by drying under reduced pressure the resin obtained after washing 10 times with 5 mL of dimethylformamide, 5 times with methanol, and 3 times with diethyl ether, sequentially. One milligram of the resin obtained was taken separately and confirmed to be negative when subjected to a Kaiser test.

(6) Synthesis of Compound B

A stirring bar and compound 12 (17.6 mg) were placed in a 10 mL glass test tube, and 1,2-dichloroethane (2.0 mL) was added and stirred gently to swell the resin. After stirring for 5 minutes, the solvent was removed by a Pasteur pipette. The test tube was then cooled using an ice bath, and a mixed solution of pyridine (3.7 μL), sulfuryl chloride (5 μL), and 1,2-dichloroethane (995 μL) prepared in a separate 30 mL Erlenmeyer flask was added and stirred gently while cooling by ice. After stirring for 1.5 hours, the solution was removed using a Pasteur pipette, dehydrated dichloromethane (2 mL) was added, and the resin compound was washed. After removing the wash solution by a Pasteur pipette, dichloromethane was again added, and compound B was obtained by washing in the same way 5 times.

### Example 3

Octaarginine derivative modification of captopril (compound 13), a compound having an SH group, was carried out in accordance with the following synthesis scheme using compound A.

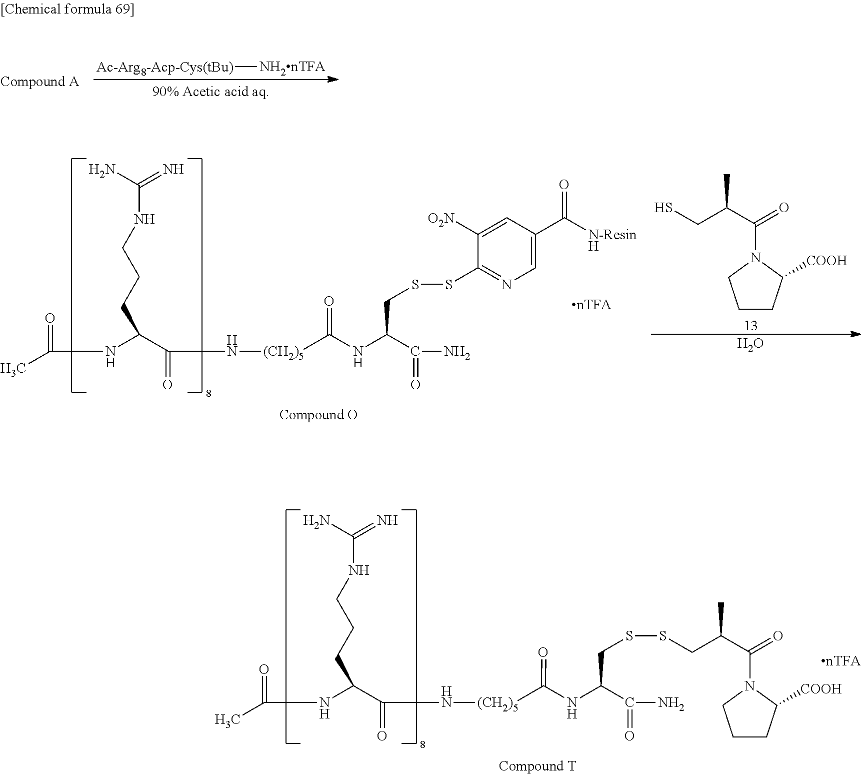

Resin: Crosslinked Polyethylene Glycol (Methyl ChemMatrix® Resin)

90% Formic acid aqueous solution (2 mL) was added to a 10 mL glass test tube containing compound A and a stirring bar while cooling by ice bath, and solvent substitution was carried out by stirring gently. After removing the wash solution by a Pasteur pipette, 90% formic acid aqueous solution was again added, and washing was repeated in the same way 5 times. An octaarginine-containing peptide Ac-Arg₈-Acp-Cys(tBu)-NH₂.TFA salt (143.37 mg) comprising 10 residues was dissolved in 90% formic acid (1 mL) in a separate 30 mL Erlenmeyer flask, and the aqueous solution obtained was added to the above 10 mL glass test tube containing compound A while cooling by ice. After stirring gently for 2 hours while cooling by ice, the solution was suctioned using a Pasteur pipette, and the unreacted octaarginine-containing peptide was recovered by freeze drying. Ultrapure water (2 mL) was added to the remaining resin, and the resin compound was washed. After removing the wash solution by a Pasteur pipette, ultrapure water was again added, and a solid phase-supported octaarginine-containing peptide compound O was obtained by repeating washing in the same way 5 times. The ice bath was removed, and an aqueous solution (500 μL) of captopril (compound 13, 0.99 mg) was added to the compound O obtained at room temperature and stirred gently. Thirty minutes after the start of the reaction, the solid support was filtered out, and it was confirmed by analyzing the solution obtained as the filtrate by reverse-phase HPLC that the peak from the raw material captopril had basically disappeared and the product had been converted into octaarginine-modified captopril (compound T) at an HPLC purity of 95%. It was also confirmed that the expected compound T had been obtained by analyzing the solution obtained by TOF-MS.

(HRMS (ES⁺) calcd for C₆₃H₁₃₃N₃₆O₁₄S₂ [M+3H]³ 580.6748. found m/z 580.6728.)

### Example 4

De novo synthesis of a disulfide peptide was carried out in accordance with the following synthesis scheme using compound A.

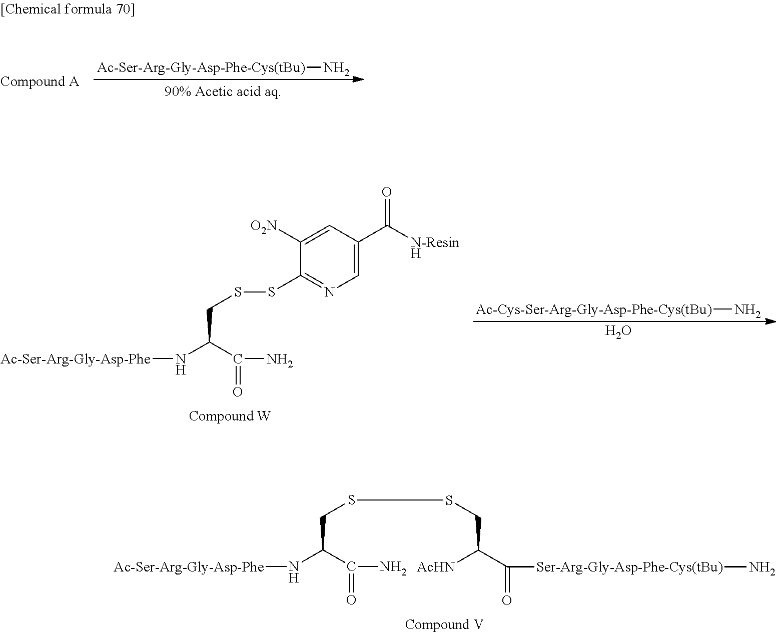

Resin: Crosslinked Polyethylene Glycol (Methyl ChemMatrix® Resin)

90% Acetic acid aqueous solution (1.5 mL) was added to a 10 mL glass test tube containing compound A (0.023 mmol) and a stirring bar while cooling by an ice bath, and solvent substitution was carried out by stirring gently. After removing the wash solution by a Pasteur pipette, 90% acetic acid aqueous solution was again added, and washing was repeated in the same way 5 times. A peptide Ac-Ser-Arg-Gly-Asp-Phe-Cys(tBu)-NH₂.TFA salt (6.18 mg) comprising 6 residues was dissolved in 90% acetic acid (0.75 mL) in a separate 30 mL Erlenmeyer flask, and the aqueous solution obtained was divided into thirds and added to the above 10 mL glass test tube containing compound A a total of three times at one hour intervals. After stirring gently for one hour while cooling by ice, the solution was suctioned off using a Pasteur pipette. Ultrapure water (2 mL) was added to the resin remaining, and the resin compound was washed. After removing the wash solution by a Pasteur pipette, ultrapure water was again added, and a solid phase-supported acetylhexapeptide compound W was obtained by repeating washing in the same way 10 times. The ice bath was removed, and an aqueous solution (0.25 mL) of a peptide Ac-Cys-Ser-Arg-Gly-Asp-Phe-Cys(tBu)-NH₂ (compound 13, 1.53 mg) comprising 7 residues was added to the compound W obtained at room temperature and stirred gently. Thirty minutes after the start of the reaction, the solid support was filtered out, and it was confirmed by analyzing the solution obtained as the filtrate by reverse-phase HPLC that the peak from the raw material acetylheptapeptide had basically disappeared and the product had been converted into a peptide (compound V) comprising 13 residues at an HPLC purity of 71%. It was also confirmed that the expected compound V had been obtained by analyzing the solution obtained by TOF-MS.

(HRMS(ES⁺) calcd for C₆₅H₁₀₀N₂₁O₂₁S₃ [M+2H]²¹ 803.8322. found m/z 803.8383.)

### Example 5

Acetylcysteine modification of captopril (compound 13), a compound having an SH group, was carried out in accordance with the following synthesis scheme using compound A.

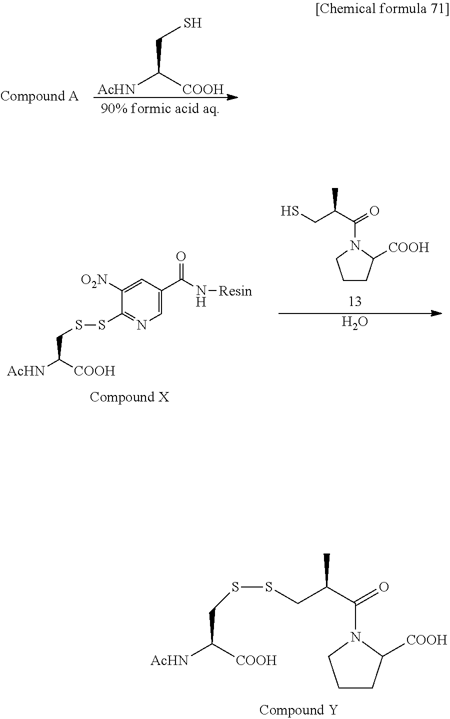

Resin: Crosslinked Polyethylene Glycol (ChemMatrix® Resin)

90% Formic acid aqueous solution (1.5 mL) was added to a 10 mL glass test tube containing compound A (0.018 mmol) and a stirring bar while cooling by ice bath, and solvent substitution was carried out by stirring gently. Alter removing the wash solution by a Pasteur pipette, 90% formic acid aqueous solution was again added, and washing was repeated in the same way 5 times. N-acetylcysteine (15.0 mg) was dissolved in 90% formic acid (1 mL) in a separate 2 mL polypropylene tube, and added to the above 10 mL glass test tube containing compound A while cooling by ice. After stirring gently for 2 hours while cooling by ice, the solution was suctioned off using a Pasteur pipette. Ultrapure water (2 mL) was added to the remaining resin, and the resin compound was washed. After removing the wash solution by a Pasteur pipette, ultrapure water was again added, and a solid phase-supported N-acetylcysteine peptide compound X was obtained by repeating washing in the same way 10 times. The ice bath was removed, and an aqueous solution (900 μL) of captopril (compound 13, 1.00 mg) was added at room temperature and stirred gently. Forty-eight hours after the start of the reaction, the solid support was filtered out, and it was confirmed by analyzing the solution obtained as the filtrate by reverse-phase HPLC that the peak from the raw material captopril had basically disappeared and the product had been converted into an acetylcysteine-captopril disulfide (compound Y) at an HPLC purity of 92%. It was also confirmed that the expected compound Y had been obtained by analyzing the solution obtained by TOF-MS.

(HRMS(ES⁺) calcd for C₁₄H₂₂N₂O₆S₂Na [M+Na]⁺ 401.0817. found m/z 401.0801.)

### Example 6

Oxytocin (compound Z), a physiologically active peptide, was synthesized in accordance with the following synthesis scheme using disulfide ligation by compound A as an example of a compound of the present invention.

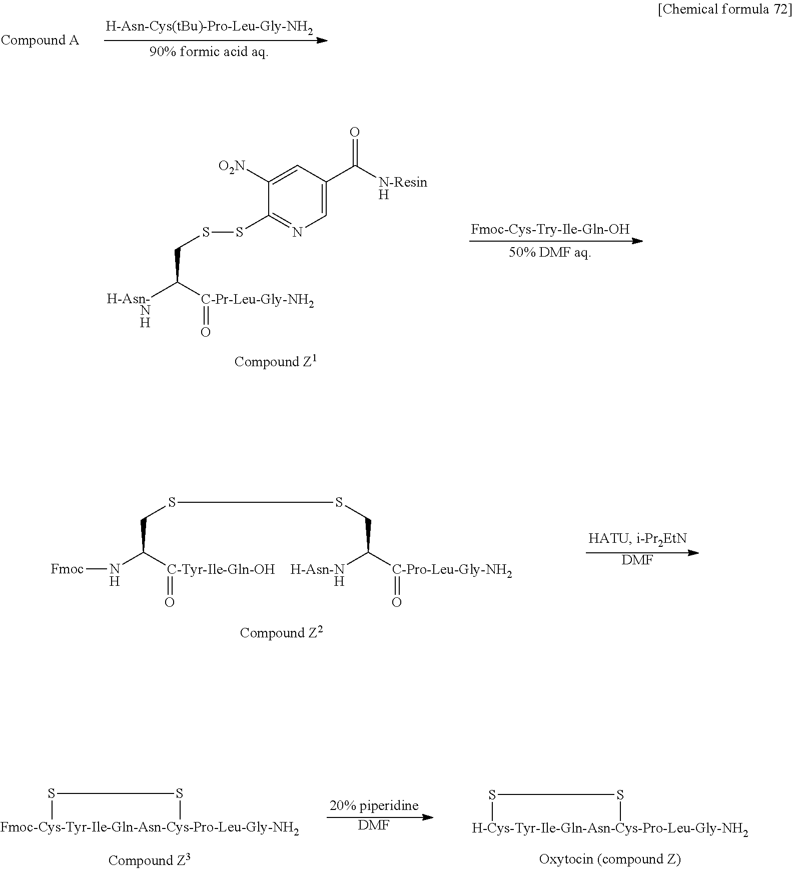

Resin: Crosslinked Polyethylene Glycol (ChemMatrix® Resin)

First, compound Z¹ was synthesized as follows. 90% formic acid aqueous solution (0.5 mL) was added to a 3 mL polypropylene column, equipped with a filter, containing compound A (0.012 mmol) while cooling by ice bath, and solvent substitution was carried out by stirring gently. After removing the wash solution by filtration, ice-cooled 90% formic acid aqueous solution (0.5 mL) was again added, and washing was repeated in the same way 5 times. Next, a 90% formic acid aqueous solution (0.248 mL) of a peptide H-Asn-Cys(tBu)-Pro-Leu-Gly-NH₂ (1.54 mg, 0.0023 mmol) was added while cooling by ice bath and, after stirring gently for two hours while cooling by ice, disappearance of the peak corresponding to H-Asn-Cys(tBu)-Pro-Leu-Gly-NH₂ was confirmed by reverse-phase HPLC (gradient: Milli-Q (0.1% TFA)/CH₃CN=95:5 to 45:55 over 25 min, flow rate: 0.9 mL/min, UV: 230 nm, column: Sunfire™ C18 5 μm, 4.6×150 mmn column).

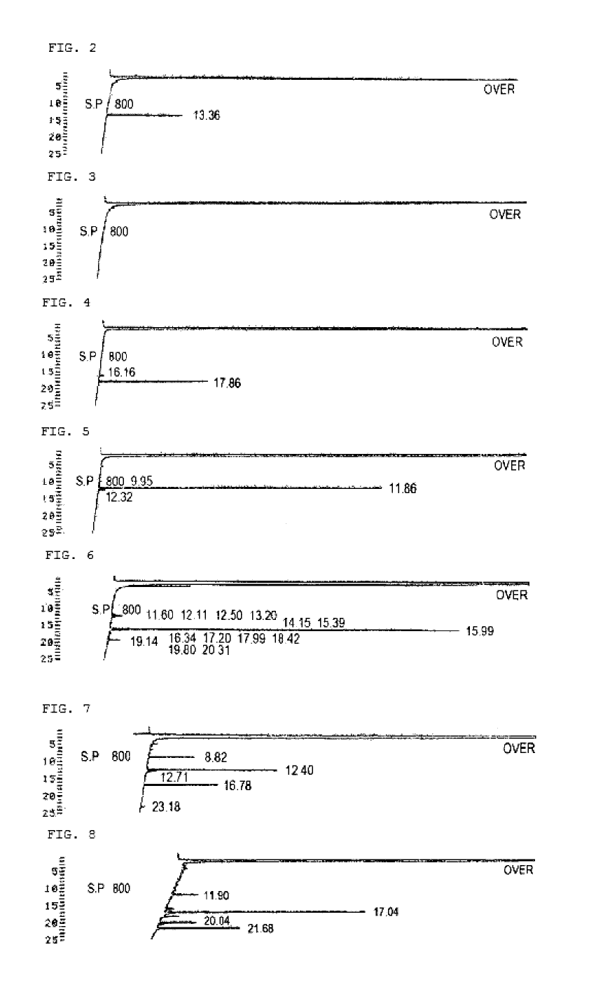

FIG. 2 shows a reverse-phase HPLC chart of a 90% formic acid aqueous solution of the peptide H-Asn-Cys(tBu)-Pro-Leu-Gly-NH₂ used in the reaction. FIG. 3 shows a reverse-phase HPLC chart of the reaction solution two hours after the start of the reaction.

The reaction solution was removed by filtration, ice-cooled ultrapure water (0.5 mL) was added, and the resin was washed by stirring gently. After removing the wash solution by filtration, ice-cooled ultrapure water (0.5 mL) was again added, and compound Z¹ was obtained by washing in the same way 5 times. Compound Z² was then synthesized using the compound Z¹ as it was as follows. The ice bath was removed from the reaction system, and a 50% N,N-dimethylformamide aqueous solution (415 μL) of a peptide Fmoc-Cys-Tyr-Ile-Gln-OH (1.43 mg, 0.0019 mmol) was added at room temperature and stirred gently. Thirty minutes after the start of the reaction, disappearance of the peak corresponding to Fmoc-Cys-Tyr-Ile-Gln-OH was confirmed by reverse-phase HPLC (gradient: Milli-Q (0.1% TFA)/CH₃CN=80:20 to 30:70 over 25 min, flow rate: 0.9 mL/min, UV: 230 nm, column: Sunfire™ C18 5 μm, 4.6×150 mmn column), and a new peak was observed at a purity of 97%.

FIG. 4 shows a reverse-phase HPLC chart of the 50% N,N-dimethylformamide aqueous solution of the peptide Fmoc-Cys-Tyr-Ile-Gln-OH used in the reaction. FIG. 5 shows a reverse-phase HPLC chart of the reaction solution 30 minutes after the start of the reaction.

The solid support was filtered out, and it was confirmed that the expected disulfide peptide compound Z² had been obtained by analyzing the solution obtained by TOF-MS (HRMS (ES⁺) calcd for C₅₈H₇₉N₁₂O₁₅S₂ [M+H]⁺ 1247.5229. found m/z 1247.5229).

Next, compound Z³ was synthesized using the compound Z² obtained. HATU (0.514 mg, 1.67 μmol) and N,N-diisopropylethylamine (0.474 μL, 2.79 μmol) were added while cooling by ice and stirring to a N,N-dimethylformamide solution (1.12 mL) of compound Z² (1.50 mg, 1.12 μmol) and stirred overnight at room temperature. After stirring overnight, part of the reaction solution was analyzed by reverse-phase HPLC (gradient: Milli-Q (0.1% TFA)/CH₃CN=80:20 to 30:70 over 25 min, flow rate: 0.9 mL/min, UV: 230 nm, column: Sunfire™ C18 5 μm, 4.6×150 mmn column), and disappearance of the peak corresponding to Z² and a new peak were observed. FIG. 6 shows a reverse-phase HPLC chart of the reaction solution after reacting overnight. It was confirmed that the expected compound Z³ had been obtained by analyzing the fraction detected at 15.99 minutes, which was the main peak, by TOF-MS (HRMS (ES⁺) calcd for C₅₈H₇₇N₁₂O₁₄S₂ [M+H]⁺ 1229.5124. found m/z 1229.5181).

Oxytocin (compound Z), which is a physiologically active peptide, was synthesized next using the compound Z³ obtained. Compound Z³ (1.52 mg, 1.24 μmol) was placed in a glass container, and a 20% piperidine/DMF solution (0.4 mL) was added at room temperature and stirred overnight at room temperature. After stirring overnight, part of the reaction solution was analyzed by reverse-phase HPLC (gradient: Milli-Q (0.1% TFA)/CH₃CH=95:5 to 45:55 over 25 min, flow rate: 0.9 mL/min, UV: 230 nm, column: Sunfire™ C18 5 μm, 4.6×150 mmn column), and disappearance of the peak corresponding to Z³ and a new peak were observed. FIG. 7 shows a reverse-phase HPLC chart of the reaction solution after reacting overnight. It was confirmed that the expected oxytocin (compound Z) had been obtained by analyzing the fraction detected at 12.40 minutes by TOF-MS (HRMS (ES⁺) calcd for C₄₃H₆₇N₁₂O₁₂S₂ [M+H]⁺ 1007.4443. found m/z 1007.4418).

### Example 7

A train peptide (compound V¹) was synthesized in accordance with the following scheme using disulfide ligation by compound A as an example of a compound of the present invention.

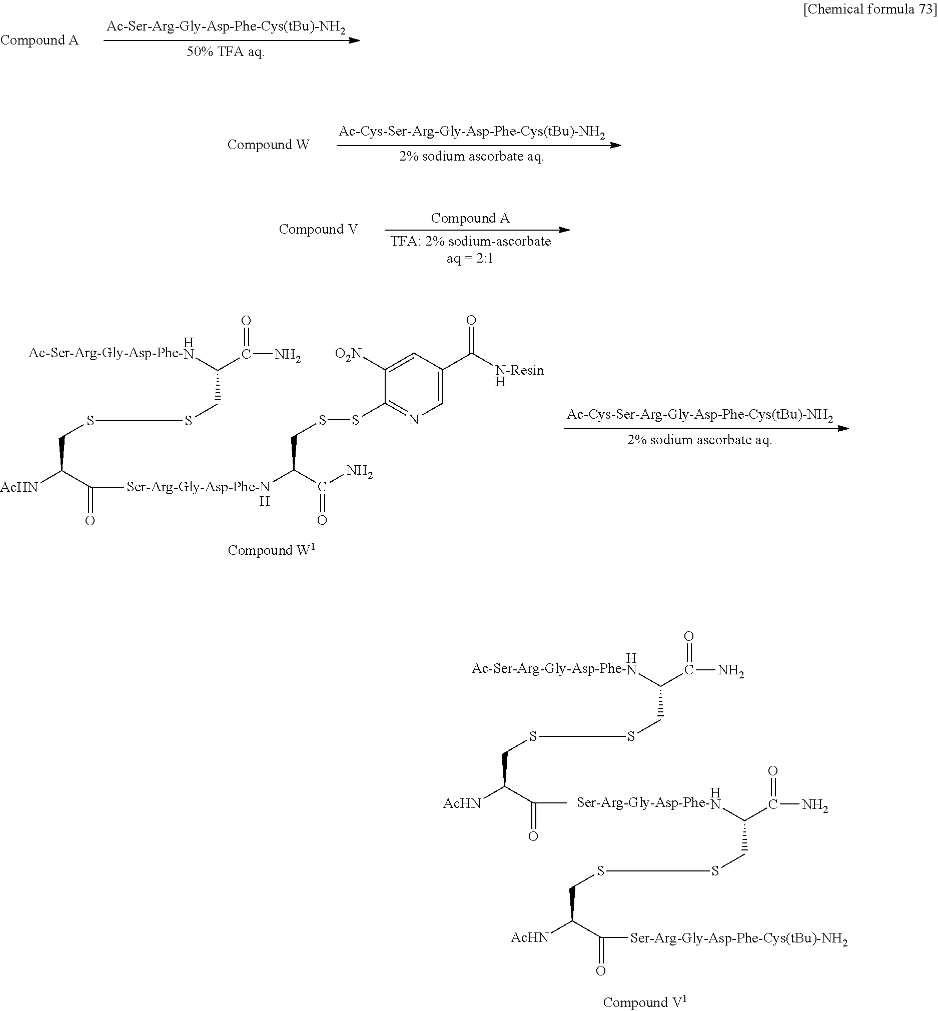

Resin: Crosslinked Polyethylene Glycol (ChemMatrix® Resin)

First, compound W was synthesized as follows.

A 50% TFA aqueous solution (250 μL) was added to a 3 mL polypropylene column, equipped with a filter, containing compound A (11.5 μmol) while cooling by ice bath, and solvent substitution was carried out by stirring gently. After removing the wash solution by filtration, ice-cooled 50% TFA aqueous solution (250 μL) was again added, and washing was repeated in the same way 5 times. A 50% TFA aqueous solution (250 μL) of a peptide Ac-Ser-Arg-Gly-Asp-Phe-Cys(tBu)-NH₂ (2.06 mg, 2.30 μmol) was then added while cooling by ice bath. After stirring gently for 2 hours while cooling by ice, the reaction solution was removed by filtration, ice-cooled 2% sodium ascorbate aqueous solution (250 μL) was added, and the resin was washed by stirring gently. After removing the wash solution by filtration, ice-cooled 2% sodium ascorbate aqueous solution (250 μL) was again added, and compound W was obtained by washing in the same way 10 times.

Compound V was then synthesized as follows using the compound W obtained as it was.

The ice bath was removed from the reaction system, and a 2% sodium ascorbate aqueous solution (250 μL) of a peptide Ac-Cys-Ser-Arg-Gly-Asp-Phe-Cys(tBu)-NH₂ (1.53 mg, 1.53 μmol) was added at room temperature and stirred gently. Thirty minutes after the start of the reaction, the solid support was filtered out, 95% TFA aqueous solution (250 μL) was added, and the resin was washed by stirring gently. Washing was repeated in the same way twice, the filtrate and wash solution were combined, and compound V was obtained as a solution. Compound W¹ was synthesized as follows using this solution of compound V as it was.

The solution of compound V was added directly to a 3 mL polypropylene column, equipped with a filter, containing separately prepared compound A (11.5 μmol) while cooling by ice and stirred for 5 hours while cooling by ice. The reaction solution was then removed by filtration, ice-cooled 2% sodium ascorbate aqueous solution (250 μL) was added, and stirred gently. After removing the wash solution by filtration, ice-cooled 2% sodium ascorbate aqueous solution (250 μL) was again added, and washing was repeated in the same way 10 times. It was confirmed using pH test paper that the wash solution had become pH=5, and compound W¹ was obtained.

Compound V¹ was synthesized as follows using the compound W¹ obtained as it was.

The ice bath was removed from the reaction system, and a 2% sodium ascorbate aqueous solution (250 μL) of Ac-Cys-Ser-Arg-Gly-Asp-Phe-Cys(tBu)-NH₂ (1.02 mg, 10.2 μmol) was added at room temperature and stirred gently. Thirty minutes after the start of the reaction, the solid support was filtered out, the filtrate was analyzed by reverse-phase HPLC (gradient: water (0.1% TFA)/CH₃CH=10:90 to 65:35 over 25 min, flow rate: 0.9 mL/min, UV: 230 nm, column: Sunfire™ C18 5 μm, 4.6×150 mm column), and disappearance of the peak corresponding to Ac-Cys-Ser-Arg-Gly-Asp-Phe-Cys(tBu)-NH₂ and a new peak were observed. It was confirmed that the expected compound V¹ had been obtained by analyzing the fraction at 17.04 minutes, which was the main peak, by TOF-MS (HRMS (ES⁺) calcd for C₉₇H₁₄₆N₃₂O₃₂S₅ [M+3H]⁺ 811.3206. found m/z 811.3179).

1. A compound represented by formula (I) or a salt thereof. 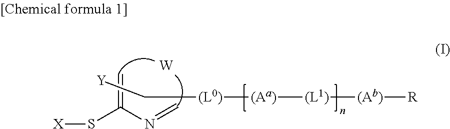
(In the formula,
W, together with other ring member atoms, forms a nitrogen-containing heterocycle selected from pyridine, pyrazine, imidazole, oxazole, thiazole, quinoline, isoquinoline, quinoxaline, phenanthroline, pteridine, or azocine,
X represents a halogen atom selected from fluorine, chlorine, bromine, or iodine,
Y represents a hydrogen atom or electron-withdrawing substituent present on the nitrogen-containing heterocycle,
R represents a polymeric support,
L⁰, L¹ may each be present independently and, when present, represent linkers having a chemically stable structure,
A^{a}, A^{b} may each be present independently and, when present, represent functional groups connecting L⁰-L¹, L¹-R, respectively, and
n represents an integer of 0-10.)

2. The compound according to claim 1, or a salt thereof, wherein A^{a}, A^{b}, when present, are each independently selected from the group consisting of alkenes, alkynes, carbonyls, esters, ethers, oxyalkylenes, amides, ureas, hydrazines, triazoles, sulfones, sulfoxides, sulfonic acid esters, sulfonamides, sulfinic acid esters, sulfinamides, piperidines, and dioxanes.

3. The compound according to claim 1 represented by formula (II), or a salt thereof, wherein the nitrogen-containing heterocycle is a pyridine ring, L¹ is not present, A^{a} is an amide group, A^{b} is not present, and n is 1. 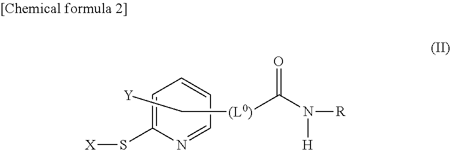
(In the formula, X, Y, R, and L⁰ are as defined in formula (I).)

4. The compound according to claim 1 represented by formula (II-a), or a salt thereof, wherein the nitrogen-containing heterocycle is a pyridine ring, A^{a} is an amide group, A^{b} is an amide group, and n is 1-5. 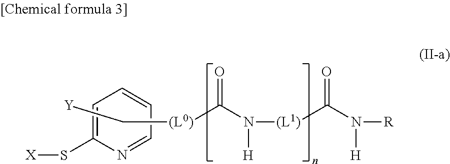
(In the formula, X, Y, R, L⁰, and L¹ are as defined in formula (I).)

5. The compound according to claim 1, or a salt thereof, wherein the electron-withdrawing substituent is a nitro group, trifluoromethyl group, or halogen.

6. The compound according to claim 1, or a salt thereof, wherein L⁰ and L¹, when present, each independently are selected from the group consisting of straight or branched C1-C20 alkylenes, C2-C20 alkenylenes, C2-C20 alkynylenes, cycloalkylenes having 3-20 carbon atoms, cycloalkenylenes having 3-20 carbon atoms, arylenes, monocyclic heteroarylenes, heterocycles, amines, amides, ethers, esters, sulfides, ketones, polyethylene glycol chains, and groups represented by formula (a) 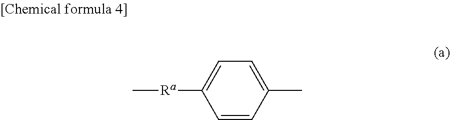
(in the formula, R^{a} represents an optionally substituted C1-C15 alkylene), and these alkylenes, alkenylenes, alkynylenes, cycloalkylenes, cycloalkenylenes, arylenes, and monocyclic heteroarylenes are optionally substituted.

7. The compound according to claim 1, or a salt thereof, wherein R is a polymeric support used in a solid-phase synthesis method.

8. The compound according to claim 7, or a salt thereof, wherein R is selected from the group consisting of polystyrene, polypropylene, polyethylene, polyether, polyvinyl chloride, dextran, acrylamide, polyethylene glycol, copolymers and crosslinked forms thereof, magnetic beads, and combinations thereof.

9. An SH group selective reactive solid-phase supported reagent containing the compound according to claim 1, or a salt thereof.

10. A method for producing a compound represented by formula (IV), wherein the method comprises
reacting a compound represented by formula (I) 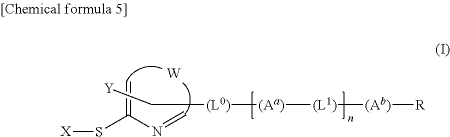
(in the formula, W, together with other ring member atoms, forms a nitrogen-containing heterocycle selected from pyridine, pyrazine, imidazole, oxazole, thiazole, quinoline, isoquinoline, quinoxaline, phenanthroline, pteridine, or azocine,
X represents a halogen atom selected from fluorine, chloride, bromine, or iodine,
Y represents a hydrogen atom or electron-withdrawing substituent,
R represents a polymeric support,
L⁰, L¹, when present, represent linkers having a chemically stable structure,
A^{a}, A^{b}, when present, represent functional groups connecting L⁰-L¹, L¹-R, respectively, and
n represents an integer of 0-10)
with a compound represented by formula (III) 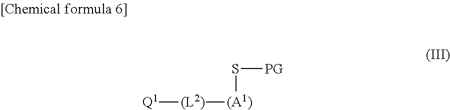
(in the formula,
Q¹ represents an organic compound,
L², when present, represents a linker having a chemically stable structure,
A¹, when present, represents a functional group having S-PG,
PG represents an SH group protecting group or hydrogen atom) to produce a compound represented by formula (IV) 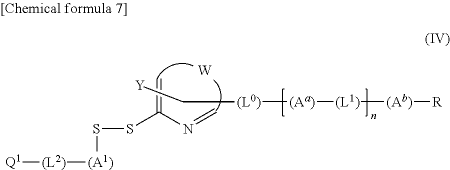
(in the formula, W, Y, R, L⁰, L¹, A^{a}, A^{b}, and n are as defined in formula (I), and Q¹, L², and A¹ are as defined in formula (III)).

11. The method according to claim 10 wherein L² is selected from the group consisting of straight or branched C1-C10 alkylenes, C2-C10 alkenylenes, C2-C10 alkynylenes, cycloalkylenes having 3-10 carbon atoms, cycloalkenylenes having 3-10 carbon atoms, arylenes, monocyclic heteroarylenes, heterocycles, amines, amides, ethers, esters, sulfides, ketones, polyethylene glycol chains, polyamides, and groups represented by formula (a) 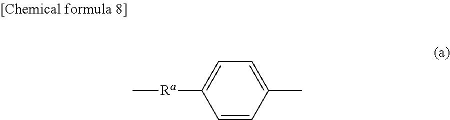
(in the formula, R^{a} represents an optionally substituted C1-C15 alkylene), and these alkylenes, alkenylenes, alkynylenes, cycloalkylenes, cycloalkenylenes, arylenes, and monocyclic heteroarylenes are optionally substituted.

12. The method according to claim 10 wherein Q¹ is selected from the group consisting of biological organic compounds, selected from amino acids, peptides, proteins, antibodies, nucleic acid bases, nucleotides or nucleosides, polymer compounds, low-molecular compounds, fluorescent labeling substances, enzyme labeling substances, biotin, chelating agents, and derivatives thereof including isotopes.

13. The method according to claim 10 wherein the SH group protecting group is selected from t-butyl, trityl, benzhydryl, benzyl, methylbenzyl, dimethylbenzyl, trimethylbenzyl, methoxybenzyl, dimethoxybenzyl, trimethoxybenzyl, nitrobenzyl, acetamidomethyl, 9-fluorenylmethyl, carbonylbenzyloxy, diphenylbenzyl, ethylcarbamoyl, picolyl, sulfonyl, or salts thereof.

14. A method for producing a compound represented by formula (VI), wherein the method comprises
reacting a compound represented by formula (IV) 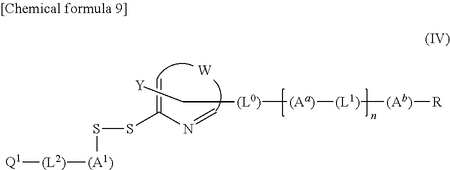
(in the formula,
W, together with other ring member atoms, forms a nitrogen-containing heterocycle selected from pyridine, pyrazine, imidazole, oxazole, thiazole, quinoline, isoquinoline, quinoxaline, phenanthroline, pteridine, or azocine,
Y represents a hydrogen atom or electron-withdrawing substituent,
R represents a polymeric support,
L⁰, L¹, L², when present, represent linkers having a chemically stable structure,
A^{a}, A^{b}, when present, represent functional groups connecting L⁰-L¹, L¹-R, respectively,
A¹, when present, represents a functional group having S-PG,
Q¹ represents an organic compound, and
n represents an integer of 0-10)
with a compound represented by formula (V) 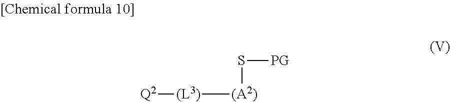
(in the formula,
Q² represents an organic compound,
L³, when present, represents a linker having a chemically stable structure, A², when present, represents a functional group having S-PG, and PG represents an SH group protecting group or hydrogen atom) to produce a compound represented by formula (VI) 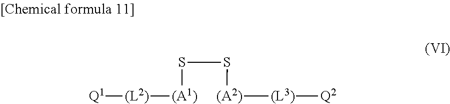
(in the formula, Q¹, Q², L², L³, A¹, and A² are as defined above).

15. The method according to claim 14 wherein the electron-withdrawing substituent is a nitro group, trifluoromethyl group, or halogen.

16. The method according to claim 14 wherein L², L³ are each independently selected from the group consisting of straight or branched C1-C10 alkylenes, C2-C10 alkenylenes, C2-C10 alkynylenes, cycloalkylenes having 3-10 carbon atoms, cycloalkenylenes having 3-10 carbon atoms, arylenes, monocyclic heteroarylenes, heterocycles, amines, amides, ethers, esters, sulfides, ketones, polyethylene glycol chains, polyamides, and groups represented by formula (a) 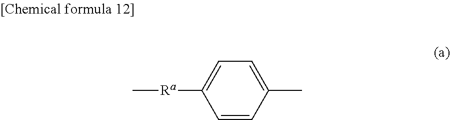
(in the formula, R^{a} represents an optionally substituted C1-C15 alkylene), and these alkylenes, alkenylenes, alkynylenes, cycloalkylenes, cycloalkenylenes, arylenes, and monocyclic heteroarylenes are optionally substituted.

17. The method according to claim 14 wherein Q¹, Q² are each independently selected from the group consisting of biological organic compounds, selected from amino acids, peptides, proteins, antibodies, nucleic acid bases, nucleotides or nucleosides, polymer compounds, low-molecular compounds, fluorescent labeling substances, enzyme labeling substances, chelating agents, biotin, and derivatives thereof including stable isotopes.

18. A method for producing a compound represented by formula (II), wherein the method comprises the following steps of:
(a) preparing a compound represented by formula (2) by reacting a compound represented by formula (1) with thionyl chloride, oxalyl chloride, dichloroalkylhydantoin, phosphorus oxychloride, or phosphorus pentachloride, 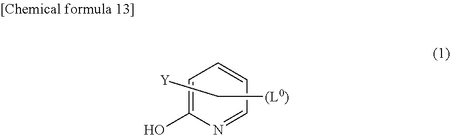
(wherein Y represents a hydrogen atom or electron-withdrawing substituent, and L⁰, when present, represents a chemically stable linker) 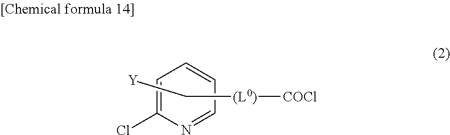
(b) preparing a compound represented by formula (3) by reacting a compound represented by formula (2) with R′OH (wherein R′ represents a C1-C10 alkyl group), 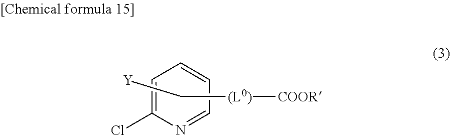
(c) preparing a compound represented by formula (4) by reacting a compound represented by formula (3) with a primary to tertiary alkylthiol under basic conditions, 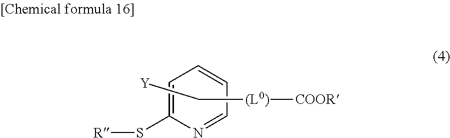
(wherein R″ represents a primary to tertiary carbon serving as a leaving group)
(d) preparing a compound represented by formula (5) by hydrolyzing a compound represented by formula (4) under basic conditions 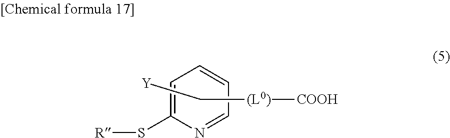
(e) preparing a compound represented by formula (6) by reacting a compound represented by formula (5) with NH₂—R (wherein R represents a polymeric support) in the presence of a base, and 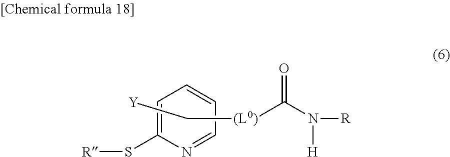
(f) preparing a compound represented by formula (II) by reacting a compound represented by formula (6) with sulfuryl chloride, chlorine gas, phosphorus oxychloride, phosphorus pentachloride, bromine, fluorinated alkyl pyridine, fluorinated quinuclidine, or iodine 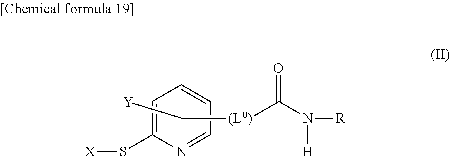
(wherein X represents a halogen atom selected from fluorine, chloride, bromine, or iodine, Y represents a hydrogen atom or electron-withdrawing substituent, R represents a polymeric support, and L⁰, when present, represents a linker having a chemically stable structure).

19. A method for producing a compound represented by formula (II-a′), wherein the method comprises the following steps of:
(g) preparing a compound represented by formula (8) by reacting a compound represented by formula (7) with NH₂—R (wherein R represents a polymeric support) in the presence of a dehydrocondensing agent, 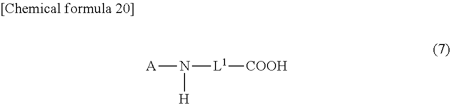
(wherein A represents an amino group protecting group having a urethane structure, and L¹ represents a linker having a chemically stable structure) 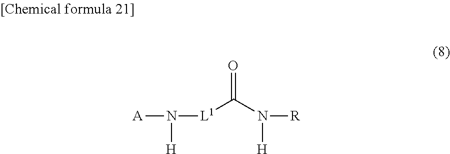
(h) preparing a compound represented by formula (9) by reacting a compound represented by formula (8) with piperidine, diethylamine, dialkylamine, trifluoroacetic acid, hydrochloric acid, or hydrogen chloride or through catalytic hydrogen reduction, 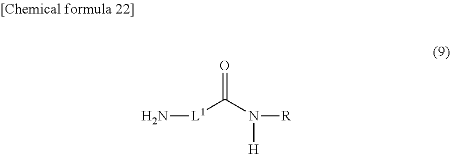
(i) preparing a compound represented by formula (10) by reacting a compound of formula (9) with a compound of formula (7) in the presence of a dehydrocondensing agent, 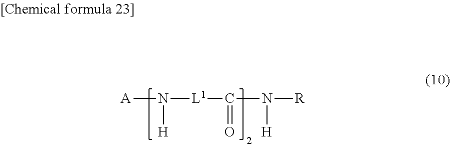
(j) preparing a compound represented by formula (11) by alternately subjecting a compound represented by formula (10) repeatedly to the procedures of steps (h) and (i) n-2 times, 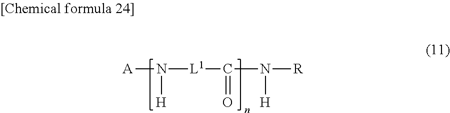
(k) preparing a compound represented by formula (12) by reacting a compound represented by formula (11) with piperidine, diethylamine, dialkylamine, trifluoroacetic acid, hydrochloric acid, or hydrogen chloride or through catalytic hydrogen reduction, 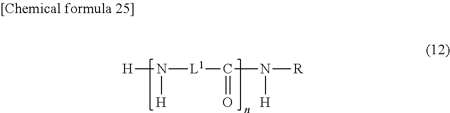
(l) preparing a compound represented by formula (13) by reacting a compound represented by formula (12) with a compound represented by formula (5) in the presence of a dehydrocondensing agent, and 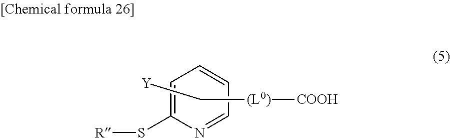
(wherein Y represents a hydrogen atom or electron-withdrawing substituent, L⁰, when present, represents a chemically stable linker, and R″ represents a primary to tertiary carbon serving as a leaving group) 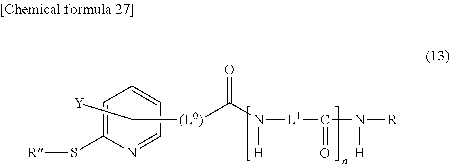
(m) preparing a compound represented by formula (II-a′) by reacting a compound represented by formula (13) with sulfuryl chloride or chlorine gas 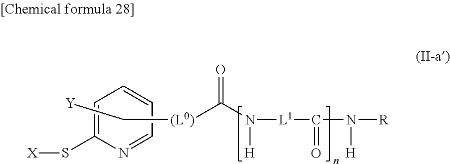
(in the formula, X represents a halogen atom selected from fluorine, chlorine, bromine, or iodine, Y represents a hydrogen atom or electron-withdrawing substituent, R represents a polymeric support, L⁰, when present, represents a chemically stable linker, L¹ represents a linker having a chemically stable structure, and n represents an integer of 1-10).


SEQUENCE LISTING (17 sequences)

SEQ ID NO: 1 - protein, 5 aa, Artificial Sequence
  analogous
NCPLG

SEQ ID NO: 2 - protein, 4 aa, Artificial Sequence
  analogous
CYIQ

SEQ ID NO: 3 - protein, 10 aa, Artificial Sequence
  analogous
RRRRRRRRXC

SEQ ID NO: 4 - protein, 6 aa, Artificial Sequence
  analogous
SRGDFC

SEQ ID NO: 5 - protein, 7 aa, Artificial Sequence
  analogous
CSRGDFC

SEQ ID NO: 6 - protein, 7 aa, Artificial Sequence
  analogous
CSRGDFC

SEQ ID NO: 7 - protein, 10 aa, Artificial Sequence
  analogous
RRRRRRRRXC

SEQ ID NO: 8 - protein, 10 aa, Artificial Sequence
  analogous
RRRRRRRRXC

SEQ ID NO: 9 - protein, 6 aa, Artificial Sequence
  analogous
SRGDFC

SEQ ID NO: 10 - protein, 6 aa, Artificial Sequence
  analogous
SRGDFC

SEQ ID NO: 11 - protein, 5 aa, Artificial Sequence
  analogous
NCPLG

SEQ ID NO: 12 - protein, 5 aa, Artificial Sequence
  analogous
NCPLG

SEQ ID NO: 13 - protein, 4 aa, Artificial Sequence
  analogous
CYIQ

SEQ ID NO: 14 - protein, 9 aa, Artificial Sequence
  analogous
CYIQNCPLG

SEQ ID NO: 15 - protein, 9 aa, Artificial Sequence
  analogous
CYIQNCPLG

SEQ ID NO: 16 - protein, 6 aa, Artificial Sequence
  analogous
SRGDFC

SEQ ID NO: 17 - protein, 7 aa, Artificial Sequence
  analogous
CSRGDFC